SEQUENCE LISTING (1 sequence)

SEQ ID NO: 1 - protein, 671 aa, Homo sapiens
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTAYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

(19) United States
(12) Patent Application Publication
(10) Pub. No.: US 2023/0056994 A1
(43) Pub. Date: Feb. 23, 2023

(54) NECROPTOSIS MODULATORS, SCREENING METHODS AND PHARMACEUTICAL COMPOSITIONS

(71) Applicant: NEURALIS SA, Liège (BE)

(72) Inventors: Mukherjee SUMEDHA, Kolkata (IN); Kumar GAURAV, Patna (IN)

(73) Assignee: NEURALIS SA, Liège (BE)

(21) Appl. No.: 17/787,080

(22) PCT Filed: Dec. 21, 2020
(86) PCT No.: PCT/EP2020/087395

§ 371 Date: Jun. 17, 2022

(30) Foreign Application Priority Data
Dec. 20, 2019 (IN) 201911053218

(51) Int. Cl. G01N 33/573; G01N 33/74; A61P 29/00; A61K 31/565; G16C 20/50

(52) U.S. Cl. CPC G01N 33/573; G01N 33/743; A61P 29/00; A61K 31/565; G16C 20/50; G01N 2500/02; G16B 15/30

(57) ABSTRACT

The present invention concerns methods to identify RIPK1 modulators capable of modulating RIPK1 activity, RIPK1 interacting molecules that modulate RIPK1 activity and pharmaceutical compositions comprising RIPK1 modulators.

### FIELD

Aspects of the invention relate to screening methods in the molecular biology field and more specifically concern screening methods to identify RIPK1 interacting molecules.

### BACKGROUND

Cell death plays a central role in a plethora of diseases. While traditional cell death pathways such as apoptosis and necrosis have been thoroughly studied, more recently discovered subclasses of cell death are less characterized. One example hereof is necroptosis, a necrotic cell death mechanism that is activated under regulated conditions but shares many morphological features with (unregulated) necrosis such as cellular swelling and plasma membrane rupture. Nevertheless, necroptosis is emerging as a central mechanism in certain diseases and during development which can be triggered by both death receptor ligands and a variety of extracellular and intracellular stimuli inducing expression or activation of these ligands (Zhou and Yuan, Necroptosis in health and diseases, Seminar in Cell & Developmental Biology, 2014). Therefore, molecules that are able to inhibit necroptosis may be of substantial therapeutic value.

It has been established that Receptor-Interacting Protein Kinase 1 (RIPK1 or RIP1) is a key effector in necroptosis. The kinase activity of RIPK1 is crucial for activation of necroptosis by death receptor ligands, which further enables activation of downstream mediators of necroptosis such as RIPK3 (or RIP3) and MLKL. Additionally, mouse model studies have revealed a close association between necroptosis and inflammation, suggesting that RIPK1 and by extension necroptosis in general may be implicated in the pathogenesis of multiple human inflammatory diseases (Orozco et al., RIPK3 in cell death and inflammation: the good, the bad, and the ugly. Immunological reviews, 2017). Therefore, targeting RIPK1, MLKL and/or RIPK3 may provide therapeutic benefits for the treatment of human diseases characterized by necroptosis and inflammation.

Necrostatins are a class of small-molecules that have been shown to inhibit necroptosis by inhibiting RIPK1. To date, necrostatins are the best known RIPK1 inhibitors. Necrostatins inhibit RIPK1 by binding to the hydrophobic pocket of the proteins located between the N- and C-lobes of its kinase domain. By this interaction, RIPK1 is locked in an inactive conformation and the protein can no longer exert its function (Xie et al., Structural basis of RIPK1 inhibition by necrostatins, Structure, 2013). However, further studies have revealed a number of limitation of necrostatins including low metabolic stability, off-target effects, suboptimal pharmacokinetics and only a modest potency to inhibit RIPK1 (Berger et al., Characterization of GSK'963: a structurally distinct, potent and selective inhibitor of RIP1 kinase, Cell Death Discovery, 2015). Necrostatin 1 (Nec 1) is a specific inhibitor of necroptosis which acts by blocking the interaction between RIPK1 and RIPK3 and down-regulated the RIPK1-RIPK3-MLKL signal pathway (Zhang et al., Cardiovasc Toxicol. 2018 August; 18(4):346-355) known for leading to necroptosis (Quarato et al., Mol Cell. 2016 Feb. 18; 61(4): 589-601; Shan et al., GENES & DEVELOPMENT. 2018 Mar. 1; 32:327-340).

WO2017/096301 discloses methods and compositions for preventing or arresting cell death and/or inflammation by modulating RIPK1 activity.

While progress is being made, there remains a currently unmet need for novel and/or improved RIPK1/RIPK3/MLKL inhibitors that have superior characteristics compared to known inhibitors.

### SUMMARY

As evidenced in the examples which illustrate certain representative embodiments of the invention, the present invention relates to methods for identifying modulators of the RIPK1/RIPK3/MLKL necroptotic pathway based on interaction with one or more specific residues in the hydrophobic pocket of RIPK1. Furthermore, the inventors have discovered several new RIPK1 interacting molecules and related pharmaceutical compositions which modulate the necroptosis pathway comprising RIPK1, RIPK3 and MLKL.

The invention therefore provides the following aspects: Aspect 1. A method for identifying modulators of Receptor-Interacting Protein Kinase 1 (RIPK1) comprising in-silico analyzing the three-dimensional structure of a candidate molecule and assessing the degree of fit of said three-dimensional structure in the hydrophobic back pocket of RIPK1, whereby an interaction of said candidate forming a hydrogen bond with amino acid residue Leu 70 of RIPK1 as defined in SEQ ID NO.1, indicates the candidate is a modulator of RIPK1.

Aspect 2. The method according to aspect 1, wherein candidate RIPK1 modulators are further selected for their ability to form one or more hydrogen bond(s) with amino acid residue Ile 154 of RIPK1 as defined in SEQ ID NO:1.

Aspect 3. The method according to aspects 1 or 2, wherein candidate RIPK1 modulators are further selected for their ability to hydrophobically interact with any of the hydrophobic amino acid residues from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO: 1.

Aspect 4. The method according to any one of aspects 1 to 3, further comprising in vivo testing of the ability of the identified candidate modulators for modulating the activity of RIPK1, RIPK3 and/or MLKL.

Aspect 5. The method according to any one of aspects 1 to 4, wherein said modulators are inhibiting RIPK1, RIPK3 and/or MLKL activity.

Aspect 6. The method according to any one of aspects 1 to 5, wherein said modulators reversibly inhibit RIPK1, RIPK3 and/or MLKL activity.

Aspect 7. The method according to any one of aspects 1 to 5, wherein said modulators irreversibly inhibit RIPK1, RIPK3 and/or MLKL activity.

Aspect 8. The method according to any one of aspects 1 to 7, wherein said candidate modulators are naturally occurring molecules.

Aspect 9. The method according to any one of aspects 1 to 8, wherein said candidate modulators are steroid compounds, more preferably estrogens.

Aspect 10. The method according to any one of aspects 1 to 9 wherein the candidate RIPK1 modulator is further screened for its activity to modulate related proteins including but not limited to MLKL and/or RIPK3.

Aspect 11. A RIPK1 modulator capable of forming a hydrogen bond with amino acid residue Leu 70 of RIPK1 as defined in SEQ ID NO:1, preferably identified according to the method of any one of aspects 1 to 10.

Aspect 12. The RIPK1 modulator according to aspect 11, which additionally forms one or more hydrogen bond(s) with amino acid residue Ile 154 of RIPK1 as defined in SEQ ID NO:1.

Aspect 13. The RIPK1 modulator according to aspects 11 or 12, which additionally hydrophobically interacts with any of the hydrophobic amino acid residues from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO: 1.

Aspect 14. The RIPK1 modulator according to any one of aspects 11 to 13, which is an inhibitor of RIPK1, RIPK3 and/or MLKL, such as a phosphorylation inhibitor of RIPK1, RIPK3 and/or MLKL.

Aspect 15. The RIPK1 modulator according to any one of aspects 11 to 14, which is a competitive inhibitor of RIPK1, RIPK3 and/or MLKL.

Aspect 16. The RIPK1 modulator according to any one of aspects 11 to 15, which is a steroid compound, more preferably an estrogen.

Aspect 17. The RIPK1 modulator according to any one of aspects 11 to 16, which is Ethinylestradiol (EE), Estradiol (E2), Estriol (E3), or Estetrol (E4).

Aspect 18. A pharmaceutical composition comprising the RIPK1 modulator according to any one of aspects 11 to 17, for use in modulating the function of RIPK1, RIPK3 and/or MLKL.

Aspect 19. The pharmaceutical composition for use according to aspect 18, for use in inhibiting the activity of RIPK1, RIPK3 and/or MLKL, such as for inhibiting phosphorylation of RIPK1, RIPK3, and/or MLKL.

Aspect 20. The pharmaceutical composition for use according to aspect 18, for use in inhibiting or preventing necroptosis.

Aspect 20. The pharmaceutical composition for use according to aspects 18 or 19, for treating or preventing tissue injury, inflammatory diseases, or degenerative diseases.

Aspect 21. A method for modulating the function of RIPK1, comprising administering a RIPK1 modulator according to aspects 11 to 17 or a pharmaceutical composition according to aspects 18 to 20, to a subject.

Aspect 22. A method of treating or preventing necroptosis, comprising administering a RIPK1 modulator according to aspects 11 to 17 or a pharmaceutical composition according to aspects 18 to 20, to a subject.

Aspect 23. A method of treating or preventing tissue injury, inflammatory diseases, or degenerative diseases, comprising administering a RIPK1 modulator according to aspects 11 to 17 or a pharmaceutical composition according to aspects 18 to 20, to a subject.

Aspect 24. Use of a RIPK1 modulator according to aspects 11 to 17 for the manufacture of a medicament for modulating the function of RIPK1, RIPK3 and/or MLKL.

Aspect 25. Use of a RIPK1 modulator according to aspects 11 to 17 for the manufacture of a medicament for the prevention or treatment of necroptosis.

Aspect 26. Use of a RIPK1 modulator according to aspects 11 to 17 for the manufacture of a medicament for the prevention or treatment of tissue injury, inflammatory diseases, or degenerative diseases.

The above and further aspects and preferred embodiments of the invention are described in the following sections and in the appended claims. The subject matter of appended claims is hereby specifically incorporated in this specification.

### BRIEF DESCRIPTION OF THE DRAWINGS

FIG. 1: Amino acid residues in the binding pocket of RIPK1 that interact with NEC1

FIG. 2: Amino acid residues in the binding pocket of RIPK1 that interact with NEC4

FIG. 3: Amino acid residues in the binding pocket of RIPK1 that interact with Estradiol

FIG. 4: Amino acid residues in the binding pocket of RIPK1 that interact with Estriol

FIG. 5: Amino acid residues in the binding pocket of RIPK1 that interact with Estetrol

FIG. 6: Effect of Necrostatin 1 (Nec 1) and Estetrol (E4) on phosphorylation state of MLKL in MCF7 cells wherein necroptosis is induced by TNF-alpha and z-VAD-fmk. **<0.01, Ordinary One-Way ANOVA, Versus TNF-alpha z-VAD-fmk induced necroptosis. The “TNF-alpha+z-VAD-fmk” bar indicates an inducement of the necroptosis pathway, indicated by an increased phosphorylation of MLKL with respect to the non-treated cells (DMSO). The bar “TNF+Z+Nec1” indicates cells wherein inducement of the necroptosis pathway is done by TNF-alpha+z-VAD-fmk in the presence of Necrostatin-1; the bar “TNF+Z+E4” indicates cells wherein inducement of the necroptosis pathway is done by TNF-alpha+z-VAD-fmk in the presence of E4. In both cases (Nec1 or E4) the MLKL phosphorylation is reduced versus the induced cells. For Nec 1 this reduction in MLKL phosphorylation is statistically relevant (*p<0.05), for the E4 treated cells, the reduction of MLKL phosphorylation is statistically relevant (**p<0.01). Statistical analysis is done by Ordinary One-Way ANOVA (Cf. Example 12).

FIG. 7: Effect of Necrostatin 1 (Nec 1) and Estetrol (E4) on phosphorylation state of MLKL in HT29 cells wherein necroptosis is induced by TNF-alpha and z-VAD-fmk. The “TNF-alpha+z-VAD-fmk” bar indicates an inducement of the necroptosis pathway, indicated by an increased phosphorylation of MLKL with respect to the non-treated cells (DMSO). The bar “TNF+Z+Nec1” indicates cells wherein inducement of the necroptosis pathway is done by TNF-alpha+z-VAD-fmk in the presence of Necrostatin-1; the bar “TNF+Z+E4” indicates cells wherein inducement of the necroptosis pathway is done by TNF-alpha+z-VAD-fmk in the presence of E4. In both cases (Nec1 or E4) the MLKL phosphorylation is reduced versus the induced cells. For Nec 1 this reduction in MLKL phosphorylation is statistically Non-Significant (NS), for the E4 treated cells, the reduction of MLKL phosphorylation is statistically relevant (**p<0.01). Statistical analysis is done by Ordinary One-Way ANOVA (Cf. Example 12).

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Amino acids are referred to herein with their full name, their three-letter abbreviation or their one letter abbreviation.

The following detailed description is not to be taken in a limiting sense. The scope of the present invention is defined by the appended claims. It is evident that disclosed embodiments may relate to both the RIPK1 modulator, methods to identify RIPK1 modulators, and pharmaceutical compositions based on RIPK1 modulators. Certain embodiments directed to the RIPK1 modulators may apply to the methods or uses described herein.

“RIPK1” or “Receptor-Interacting Protein Kinase 1”, also known as “RIP1”, “Receptor-Interacting protein 1”, “Cell death protein RIP”, and “Receptor (TNFRSF)-Interacting Serine-Threonine Kinase 1” is a family member of the receptor-interacting protein (RIP) family of serine/threonine protein kinases which plays a role in both cell survival and cell death. In cell death, RIPK1 is connected to apoptosis and necroptosis. Cell survival pathways where RIPK1 plays a role in include NF-κB, Akt, and c-Jun N-terminal kinase (JNK). It is also involved in developmental regulation. The RIPK1 protein comprises three main domains: an N-terminal serine/threonine kinase domain (KD), a C-terminal death domain (DD), and a central intermediate domain (ID). The kinase domain plays a central role in the functionalities of RIPK1 in cell survival and necroptosis induction. It is characterized by a canonical kinase fold containing an N-lobe, C-lobe, and an intervening hydrophobic pocket. The N-lobe comprises an antiparallel, five-stranded f sheet and an activation helix. The C-lobe contains six a helices and a pair of β strands. Necrostatins interact with the kinase domain and inhibit its kinase function. Finally, the death domain displays homology to death domains of other receptors including those of Fas, TRAILR2, TNFR1, TRAILR1, TRADD and FADD to which it can bind and form oligomers. The intermediate domain is involved in NF-κB activation and Receptor-interacting protein Homotypic Interaction Motif (RHIM)-dependent signalling.

The main set of genes that have shown to be involved in necroptosis, or mediating necroptosis, i.e. RIPK1, RIPK3, and MLKL, are not found in primitive organisms. While RIPK1 orthologs have shown to be present in most vertebrate species and mammals, this is not the case for more primitive organisms including nematodes or flies. Likewise, RIPK3 and MLKL only occur in certain vertebrates and mammals, but not in some Craniata clades and other species. In mammals, the complete Carnivora order lacks the MLKL gene, whereas the infraclass of Marsupialia lacks both RIPK3 and MLKL genes. These observations argue against the hypothesis that necroptosis has evolved as an essential host defense mechanism (Dondelinger et al., An evolutionary perspective on the necroptotic pathway, Trends in Cell Biology, 2016).

The term “necroptosis”, or “controlled necrosis” refers to a programmed form of necrosis that may be activated in response to the stimulation of death receptors by their cognate ligands in absence of caspase activity, the latter being an essential mediator of apoptosis. Morphological features of necroptosis closely resemble those of necrosis and include early plasma permeabilization, swelling of organelles, an expanded nuclear membrane, and chromatin condensation. Necroptosis is initiated by binding of Tumor Necrosis Factor (TNF) to its membrane receptor, the Tumor Necrosis Factor Receptor (TNFR) which leads to recruitment of death domain protein TRADD and subsequent recruitment of RIPK1. When active Caspase 8 is not present, RIPK1 and RIPK3 will auto- and transphosphorylate each other. This leads to the formation of a microfilament-like complex termed the necrosome. Once formed, the necrosome will activate MLKL by phosphorylation and polymerization. MLKL is a pro-necroptotic protein that causes the necrosis phenotype by insertion into the bilipid membranes of organelles and the plasma membrane which will expulse cellular contents, including Damage Associated Molecular Patterns (DAMPS) to the extracellular environment. Hence, measurement of phosphorylation of RIPK1, RIPK3 and/or MLKL is a good measure for necrosis in cells or tissues.

A considerable overlap between the apoptosis and necroptosis has been described. Amongst others, Caspase 8 and TNFR are involved in both forms of cell death. In addition, RIPK1 can also govern both apoptosis and necroptosis dependent of post-translational modifications mediated by other signalling proteins (Vanden Berghe et al. Regulated necrosis: the expanding network of non-apoptotic cell death pathways, Nature Reviews, 2014). Multiple other forms of regulated cell death have been identified in addition to necroptosis, and these include pyroptosis, ferroptosis, parthanatos, cyclophilin D-dependent necrosis, ferroptosis, oxytosis, and NETosis. It is known to a person skilled in the art that the pathways leading to different mechanisms of cell death do not function in isolation but are highly interconnected and subject to crosstalk (Conrad et al. Regulated necrosis: disease relevance and therapeutic opportunities). Over time, necroptosis has been shown to be involved in a plethora of biological phenomena, including acting as a checkpoint during embryonic development able to initiate abortion of embryos that have severe developmental defects. Furthermore, necroptosis has been reported to be involved in many human diseases through its role in mediating cell death and inflammation, including but not limited to ischemic brain disease, kidney diseases, heart injuries, immunodeficiency associated with defective caspase-8, multiple sclerosis (MS), amyotrophic lateral sclerosis (ALS), LUBAC deficiency syndrome, Alzheimer's disease (AD), A20 and Abin-1 deficiency-associated immunopathologies, NEMO deficiency diseases, and wound healing.

Further study of MLKL has shown that Thioredoxin-1 (Trx1), an oxidoreductase, is reported to bind with MLKL and maintain MLKL at a reduced state under basal conditions, hereby effectively blocking necroptosis by preventing MLKL disulfide bond formation and polymerization. This suggests that Trx1 may play an important role in negative regulation of MLKL activation. However, necroptosis is not sensitive to the addition of reducing agents in many cellular model systems, a critical feature of necroptosis that distinguishes it from other forms of necrosis, such as ferroptosis (Shan et al., Necroptosis in development and diseases, Genes and Development, 2018).

The “necroptosome”, or the “necroptosis complex” as used herein is an assembly a supramolecular construction formed by RIPK1 and RIPK1 activation, interaction and subsequent recruitment and/or activation of RIPK3 and/or MLKL (through phosphorylation). Based on the latest hypotheses, the necroptosome is crucial for propagation of the necroptosis signalling to the mitochondria.

“Necrostatins” as defined herein are a class of allosteric small-molecule inhibitors of RIPK1 kinase activity capable of blocking necroptosis. Necrostatins bind RIPK1 in a hydrophobic pocket and hereby lock RIPK1 in an inactive conformation. Structural studies (cf. Example 2) have found that Necrostatin 1 shows hydrogen bonding with the side chains of Lys 45 and Asp 156. In addition, Necrostatin 1 shows hydrophobic interaction with Val 76, Leu 78, Leu 90, Val 91, Met 92, Ile 43, Leu 157, Phe 162 and Ala 155. Nec-4 forms a single hydrogen bond with the side chain of Asp 156, and hydrophobic interactions with Ser 161, Lys 45, Phe 162, Met 67, Val 76, Leu 78, Met 92, Leu 90, Leu 70, Val 75, Ala 155, Ile 154 and Leu 129. Necrostatin 1 (Nec 1) is a specific inhibitor of necroptosis which acts by blocking the interaction between RIPK1 and RIPK3 and down-regulated the RIPK1-RIPK3-MLKL signal pathway (Zhang et al., Cardiovasc Toxicol. 2018 August; 18(4):346-355). Nec 1 is thought to act on RIPK1 activity by inhibiting the phosphorylation of RIPK1, which leads to downstream effects on activation/phosphorylation of RIPK3 and/or MLKL.

“Molecular docking” is indicative for a method able to predict the preferred orientation of one molecule to a second when bound to each other to form a stable complex, which may subsequently provide information regarding binding affinities or association strength. Molecular docking finds its merits in predicting the type of signal that is produced.

“Hydrophobic”, or “hydrophobicity” as used herein describes a physical property of a molecule that is characterized by an absence of attraction to water. Commonly accepted hydrophobicity values of amino acids are known to a person skilled in the art. In addition, the hydrophobic effect in amino acid interactions, i.e. the tendency of hydrophobic amino acids to aggregate in an aqueous solution to minimize exposure to water molecules can be considered common knowledge. The hydrophobic effect can be observed in intramolecular interactions such as during protein folding, aiding in formation of the three-dimensional structure of a polypeptide or protein, or in intermolecular interactions such as substrate-binding of enzymes.

“Phosphorylation” as used herein is a term used to describe attachment of a phosphoryl group to a substrate, which is the opposite of dephosphorylation. Protein phosphorylation is among the most abundant post-translational modifications in eukaryotes. Certain enzymes including RIPK1 and RIPK3 termed kinases catalyse transfer of phosphate groups from high-energy, phosphate-donating molecules such as adenosine triphosphate (ATP) to specific substrates. Kinases are part of the large family of phosphotransferases. The phosphorylation state of a molecule, regardless whether it classifies as a protein, lipid, or carbohydrate, may affect its activity, reactivity, and interactions it engages in. Therefore, kinases are amongst others critical in metabolism, cell signalling, protein regulation, cellular transport, secretory processes. MLKL is a so-called pseudokinase, but its activity is also regulated though phosphorylation, i.e. phosphorylated MLKL is the active form.

The reference (i.e. canonical) human RIPK1 protein sequence is annotated under Uniprot (www.uniprot.org) accession number Q13546 and the crystal structure of RIPK1 in complex with known inhibitor necrostatin-4 is available in the Protein Data Bank (PDB, www.rcsb.org) under identifier 4ITJ. Further information regarding the human annotated RIPK1 gene is available under NCBI Genbank (www.ncbi.nlm.nih.gov/gene) gene ID 8737. By means of example the canonical amino acid sequence of RIPK1 is reproduced below (SEQ ID NO: 1):

MQPDMSLNVIKMKSSDFLESAELDSGGFGKVSLCFHRTQGLMIMKTVYKG
PNCIEHNEALLEEAKMMNRLRHSRVVKLLGVIIEEGKYSLVMEYMEKGNL
MHVLKAEMSTPLSVKGRIILEIIEGMCYLHGKGVIHKDLKPENILVDNDF
HIKIADLGLASFKMWSKLNNEEHNELREVDGTAKKNGGTLYYMAPEHLND
VNAKPTEKSDVYSFAVVLWAIFANKEPYENAICEQQLIMCIKSGNRPDVD
DITEYCPREIISLMKLCWEANPEARPTFPGIEEKFRPFYLSQLEESVEED
VKSLKKEYSNENAVVKRMQSLQLDCVAVPSSRSNSATEQPGSLHSSQGLG
MGPVEESWFAPSLEHPQEENEPSLQSKLQDEANYHLYGSRMDRQTKQQPR
QNVAYNREEERRRRVSHDPFAQQRPYENFQNTEGKGTAYSSAASHGNAVH
QPSGLTSQPQVLYQNNGLYSSHGFGTRPLDPGTAGPRVWYRPIPSHMPSL
HNIPVPETNYLGNTPTMPFSSLPPTDESIKYTIYNSTGIQIGAYNYMEIG
GTSSSLLDSTNTNFKEEPAAKYQAIFDNTTSLTDKHLDPIRENLGKHWKN
CARKLGFTQSQIDEIDHDYERDGLKEKVYQMLQKWVMREGIKGATVGKLA
QALHQCSRIDLLSSLIYVSQN

Accordingly, an aspect of the invention is directed to a method for identifying modulators of RIPK1 identified by SEQ ID NO:1 comprising in silico analysis of the three-dimensional structure of a candidate molecule and assessing the degree of fit of said three-dimensional structure in the hydrophobic back pocket of RIPK1, whereby an interaction of said candidate forming a hydrogen bond with amino acid residue Leu 70 of RIPK1 defined by SEQ ID NO: 1, indicates the candidate is a modulator of RIPK1.

The term “modulator” as used herein refers to a molecule that influences the effects of a primary ligand that directly activates or deactivates the function of one or more target proteins. The precise modulatory characteristics of a modulator are interdependent with the ternary complex formed consisting of the target protein, modulator, and primary ligand. The principal binding site of a modulator is often termed the orthosteric site, which may be for example the active site of an enzyme where it engages in a binding with (a) substrate(s). Additionally, modulators may exert their activity by binding to a second binding site, annotated the allosteric binding site. In certain embodiments, the modulator may be considered an allosteric modulator. Allosteric modulators are molecules which influence the effects of a primary ligand that directly activates or deactivates the function of a target protein. Allosteric modulators stabilize a conformation of the protein structure that affects either binding or efficacy of the primary ligand. In certain embodiments, the method for identifying modulators of RIPK1 is directed to the identification of molecules that down-regulate RIPK1 activity. In alternative embodiments, the method for identifying modulator of RIPK1 is directed to the identification of molecules that up-regulate RIPK1 activity. In certain embodiments, the method for identifying modulators of RIPK1 is directed to the identification of RIPK1 binding molecules that bind RIPK1 but do not directly affect RIPK1 activity. In certain embodiments the method for identifying modulator of RIPK1 is directed to the identification of the activation state of downstream molecules in the necroptosis pathway, such as RIPK3 and MLKL, e.g. based on their phosphorylation status, wherein phosphorylated forms indicate active forms.

“In silico analysis” as defined herein is indicative for an analysis conducted by a computing system or by use of a computer simulation system. Molecular docking software has been described to explore the behaviour of molecules in the binding site of a target protein. Molecular docking software optionally allows assessing the druggability of compounds and their specificity against a particular target. Molecular docking software allows searching for complementarities between shape and/or electrostatics of binding sites surfaces and ligands. Molecular docking process can be separated into two major steps: searching and scoring. Non-limiting examples of molecular docking tools and programs include DOCK, AutoDock, FlexX, Surflex, GOLD, ICM, Glide, Cdocker, LigandFit, MCDock, FRED, MOE-Dock, LeDock, RDock, UCSF Dock, FRODOCK, ZDOCK, HEX, DOT, MEGADOCK, SOFTDOCK, BiGGER, SKE-DOCK, MolFit FFT, PIPER, F²DOCK, SDOCK, Cell-Dock, FTDock, MS-DOCK, FLOG, PAS-Dock (Protein Alpha Shape-Dock), TagDock, LZerD, PatchDOCK, MEMDOCK, GAPDOCK, SymmDock, INTELEF, pyDockTET, HADDOCK, SwarmDock, PIE-Dock, ICM, QXP, Affinity, AutoDock Vina and PSOVina, SODOCK, PLANTS, ParaDockS, FIPSDock, GalaxyDock, FLIPDock, GroupBuild, LEGEND, CLIX, LUDI, HOOK, SLIDE, MoDock, Hammerhead, DugScore, DOCKVISION, and eHiTs as described in the art (Pagadala et al., Software for molecular docking: a review, Biophysical Reviews, 2017).

Over time, two different popular molecular docking approaches have been developed, a first being molecular docking based on shape complementarity or geometric matching, and a second being based on simulating the docking process whereby ligand-protein pairwise interaction energies are calculated. In certain embodiments, the molecular docking method used herein is based on shape complementarity. In alternative embodiments, the molecular docking method used herein is based on simulation.

The term “hydrogen bond” as used herein, also known as H-bond, refers to a primarily electrostatic force of attraction between hydrogen of a hydrogen bond donor and another electronegative atom comprising a lone pair of electrons, which is termed the hydrogen bond acceptor. Hydrogen bonds may be intermolecular or intramolecular, although mainly intermolecular hydrogen bonds are envisaged when hydrogen bonds are described herein. In certain embodiments, the hydrogen bond may be a dihydrogen bond. The term “hydrogen bond” in the context of the invention should be interpreted in accordance with the IUPAC definition which reads: “The hydrogen bond is an attractive interaction between a hydrogen atom from a molecule or a molecular fragment X—H in which X is more electronegative than H, and an atom or a group of atoms in the same or a different molecule, in which there is evidence of bond formation”. It is further known that the attractive interaction can arise from some combination of electrostatics (multipole-multipole and multipole-induced multipole interactions), covalency (charge transfer by orbital overlap), and dispersion (London forces), of which the relative importance will depend on the specific system. It is further evident to a person skilled in the art that hydrogen bonds may vary in strength, mainly from 1 kJ/mol to 161.5 kJ/mol. It is further established that the strength of hydrogen bonds may be derived from studying equilibria between conformers with hydrogen bonds and conformers without hydrogen bonds. In proteins, hydrogen bonds are typically formed between the backbone oxygens and amide hydrogens.

It has been described that hydrogen bonding is one of the components of Lipinski's rule of five, a commonly used rule of thumb to evaluate the likeliness of a chemical compound with a pharmacological or biological activity to be suitable as a drug suitable for oral administration in humans. Lipinksi's rule of five and exceptions to the rule have been described in detail in the state of the art (Lipinski et al. Lead- and drug-like compounds: the rule-of-five revolution, Drug Discovery today: Technologies, 2004 and Lipinksi et al. Experimental and computational approaches to estimate solubility and permeability in drug discovery and development settings, Advanced Drug Delivery Reviews, 2012). In certain embodiments, the methods as described herein to identify modulators of RIPK1 further comprise a step to select for RIPK1 modulators adhering to at least two components of Lipinski's rule of five, preferably three components, preferably four components. In alternative embodiments, the methods further include a step to select for RIPK1 modulators adhering to other rules that predict drug likeness known in the art such as the Ghose filter, the Veber's rule or the rule of three (Ghose et al. A knowledge-based approach in designing combinatorial or medicinal chemistry libraries for drug discovery. 1. A qualitative and quantitative characterization of known drug databases, Journal of combinatorial chemistry, 1999; Veber et al. Molecular properties that influence the oral bioavailability of drug candidates, Journal of Medicinal Chemistry, 2002; and Congreve et al. “A ‘rule of three’ for fragment-based lead discovery?”, Drug Discovery Today, 2003 respectively).

“Degree of fit”, alternatively indicated by “goodness of fit” in the art, is a means to indicate the likelihood that a certain pose (i.e. a candidate binding mode) represents a favourable binding interaction and allows ranking of different ligands relative to each other. In certain embodiments, the degree of fit of the three-dimensional structure in the hydrophobic back pocket of RIPK1 may be expressed with a numerical value. In certain embodiments the degree of fit may be expressed as a score. In certain embodiments, the degree of fit is correlated to the amount of favourable interactions between RIPK1 and a candidate modulator, such as but by no means limited to the number of hydrogen bonds and/or hydrophobic contacts.

In certain embodiments, the method includes a ranking step of candidate RIPK1 modulators based on in silico prediction of the likelihood that a hydrogen bond is formed with residue Leu 70 of RIPK1 as defined by SEQ ID NO: 1, wherein RIPK1 candidate modulators with a higher likelihood to form a hydrogen bond with residue Leu 70 of RIPK1 as defined by SEQ ID NO: 1 are attributed a higher ranking than any candidate RIPK1 modulator that has a predicted likelihood to form a hydrogen bond with any other amino acid residue of RIPK1. In certain embodiments, the method selects candidate RIPK1 modulators that are able to form at least one hydrogen bond with amino acid residue Leu 70. In certain embodiments, the method further selects candidate RIPK1 modulators that are able to form at least two hydrogen bonds with amino acid residue Ile 154. In certain embodiments, the method selects candidate RIPK1 modulators that are able to form at least one hydrogen bond with amino acid residue Leu 70 and two hydrogen bonds with amino acid residue Ile 154. In further embodiments, the method comprises inclusion of additional parameters to construct a ranking of candidate RIPK1 modulators, such as but by no means limited to size of the candidate RIPK1 modulator, half-life time of the candidate RIPK1 modulator, toxicity of the RIPK1 candidate modulator including immunogenicity of the RIPK1 candidate modulator. In certain embodiments, the degree of fit of the three-dimensional structure in the hydrophobic back pocket of RIPK1 may be expressed with a numerical value. In certain embodiments the degree of fit may be expressed as an absolute score. In alternative embodiments, the degree of fit may be expressed as score relative to the degree of fit of one or more Necrostatins such as Necrostatin-1, Necrostatin-1s, necrosulfamide or etanercept. In certain embodiments, the method comprises selecting candidate RIPK1 modulators that upon binding change the conformation of RIPK1. In alternative embodiments, the method comprises selecting candidate RIPK1 modulators that upon binding change the conformation of RIPK1 to an inactive conformation. In certain embodiments the method comprises selecting candidate RIPK1 modulators that upon binding change the half-life of RIPK1. In further embodiments the method comprises selecting candidate RIPK1 modulators that upon binding decrease the half-life of RIPK1. In alternative embodiments, the method comprises selecting candidate RIPK1 modulators that decrease the half-life of RIPK1. In certain embodiments the method comprises selecting candidate RIPK1 modulators that upon binding cause RIPK1 degradation.

In certain embodiments, the RIPK1 modulators identified by the methods described herein are selected from a group of naturally occurring molecules. A skilled person appreciated that “naturally occurring” molecules, or natural products are chemical compounds or substances that are produced by a living organism. Alternative, yet non-limiting terms that may be used interchangeably with “naturally occurring” include “wild type”, “unmodified”, or “standard”. In the context of the embodiment, compounds that not found in nature, or are not produced by organisms found in nature, are not considered naturally occurring molecules. Furthermore, the term “naturally occurring” does not exclude compounds that are typically synthesized in vitro by chemical processes but nevertheless may also be found in nature, or may be the result of a naturally occurring synthesis pathway found in organisms that can be identified in nature, i.e. not limited to organisms that have been generated or produced by any kind of human manipulation or intervention. Alternatively, a skilled person is aware naturally occurring molecules can also be chemically synthesized, either by partial synthesis or total synthesis. In certain embodiments, the RIPK1 modulators identified by the methods described herein are selected from a group of molecules naturally occurring in mammals. In certain embodiments, the RIPK1 modulators identified by the methods described herein are selected from a group of molecules naturally occurring in humans. In alternative embodiments, the RIPK1 modulators identified by the methods described herein may be selected from a group or library of chemically synthesized synthetic molecules.

Methods and tools to verify sequence homology, sequence similarity or sequence identity between different sequences of amino acids or nucleic acids are well known to a person skilled in the art and include Protein BLAST, ClustalW2, SIM alignment tool, TranslatorX and T-COFFEE. The percentage of identity between two sequences may show minor differences depending on the algorithm choice and parameters. The term “sequence identity” as used herein refers to the relationship between sequences at the nucleotide (or amino acid) level. The expression “% identical” is determined by comparing optimally aligned sequences, e.g. two or more, over a comparison window wherein the portion of the sequence in the comparison window may comprise insertions or deletions as compared to the reference sequence for optimal alignment of the sequences. The reference sequence does not comprise insertions or deletions. A reference window is chosen and the “% identity” is then calculated by determining the number of nucleotides (or amino acids) that are identical between the sequences in the window, dividing the number of identical nucleotides (or amino acids) by the number of nucleotides (or amino acids) in the window and multiplying by 100.

Unless indicated otherwise, the sequence identity is calculated over the whole length of the reference sequence. A skilled person is aware of the related, yet different interpretations in the art of the terms “similarity”, “homology”, and “identity” (Pearson, An introduction to sequence similarity (“homology”) searching, Current protocols in bioinformatics, 2014).

In certain embodiments, the amino acid sequence of RIPK1 as used by the method has at least 80% identity to the amino acid sequence of RIPK1 from Homo sapiens [SEQ ID NO: 1] based on the total length of the amino acid sequence of the enzyme, preferably about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%. In further embodiments, the method identifies isoform or splice-variant specific candidate RIPK1 modulators.

In certain embodiments, the method comprises further selecting the candidate RIPK1 modulators for their ability to form a hydrogen bond with amino acid residue Leu 70 of RIPK1 as defined in SEQ ID NO: 1.

In certain embodiments, the method selects candidate RIPK1 modulators that are able to form at least one hydrogen bond with amino acid residue Leu 70 and at least one hydrogen bond with amino acid residue Ile 154 of RIPK1. In certain embodiments, the method selects candidate RIPK1 modulators that are able to form at least one hydrogen bond with amino acid residue Leu 70 and at least two hydrogen bonds with amino acid residue Ile 154 of RIPK1. In certain embodiments, the method includes a ranking step of candidate RIPK1 modulators based on in silico prediction of the likelihood that at least one hydrogen bond is formed with residue Leu 70 and Ile 154 of RIPK1 as defined by SEQ ID NO: 1, wherein RIPK1 candidate molecules with a higher likelihood to form at least one hydrogen bond with residue Leu 70 of RIPK1 as defined by SEQ ID NO: 1 are attributed a higher ranking. In certain embodiments, the ranking step is a combination of a representative score indicating the likelihood for forming a hydrogen bond with Leu 70 and the likelihood for forming a hydrogen bond with Ile 154 of RIPK1 as defined by SEQ ID NO: 1. In certain embodiments, the combination is made by summation of both representative scores. In alternative embodiments, the combination is made by first transforming both representative scores with a separate coefficient.

In certain embodiments, the method comprises further selecting the candidate RIPK1 modulators capable of forming at least one hydrogen bond with Leu 70 for their ability to hydrophobically interact with one or more of the hydrophobic amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1.

“Hydrophobic amino acids” as used herein are indicative for amino acids that have hydrophobic side chains. Naturally occurring amino acids with a hydrophobic side chain are Gly, Ala, Val, Leu, Ile, Pro, Met, Phe and Trp. Hydrophobic interactions are mediated by the hydrophobic effect, which can be interpreted as the tendency of certain molecules to avoid contact with water (also known in the art as hydrophobes). The hydrophobic effect plays a major role in the folding of proteins. It is further known to a person skilled in the art that the hydrophobic effect is temperature dependent and can be quantified by measuring partition coefficients of non-polar molecules between water and non-polar solvents, which can be experimentally derived by for example calorimetry.

In certain embodiments, the method comprises selecting the candidate RIPK1 modulators for their ability to form a hydrogen bond with amino acid residue Leu 70 and hydrophobically interact with one or more amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1. In further embodiments, the method comprises selecting the candidate RIPK1 modulators for their ability to form one or more hydrogen bonds with amino acid residue Ile 154 and hydrophobically interact with any one or more amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO: 1. In yet further embodiments, the method comprises selecting the candidate RIPK1 modulators for their ability to form one or more hydrogen bonds with amino acid residue Leu 70 and Ile 154 and hydrophobically interact with any one or more amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO: 1.

In certain embodiments, the method comprises selecting the candidate RIPK1 modulators for their ability to hydrophobically interact with hydrophobic amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1, with the proviso that a hydrogen binds with Leu 70 have been predicted by the method in a previous step. In certain embodiments, the method comprises selecting the candidate RIPK1 modulators for their ability to hydrophobically interact with hydrophobic amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO: 1, with the proviso that one or more hydrogen bonds with both Ile 154 and Leu 70 have been predicted by the method in a previous step. In certain embodiments, the method comprises selecting the candidate RIPK1 modulator for their ability to hydrophobically interact with at least one, preferably at least two, at least three, at least four, at least five, at least six, at least seven, at least eight, at least nine, at least ten, at least eleven, or all twelve of the hydrophobic amino acids selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO: 1.

In certain embodiments, the method comprises selecting the candidate RIPK1 modulators for their ability to form at least one hydrogen bond with Leu 70 and hydrophobically interact with at least two additional amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1. In certain embodiments, the method comprises selecting the candidate RIPK1 modulators for their ability to form at least one hydrogen bond with both Leu 70 and Ile 154 and hydrophobically interact with at least two additional amino acid residues of any one or more amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1. In certain embodiments, a formed hydrogen bond with Ile 154 is considered to be preferred to any hydrophobical interaction with any one or more amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1.

In certain embodiments, the method further comprises in vivo testing of the ability of the identified candidate modulators for modulating the activity of RIPK1 or its downstream targets in the necroptosis pathway such as RIPK3 and/or MLKL.

The term “in vivo testing” as used herein refers to tests or experiments that are conducted on whole living organisms or living cells. Non-limiting examples of organisms include animals, humans and plants. In embodiments where the in vivo testing is performed on living cells part of a cell culture, the living cells used for in vivo testing may be derived from any living organism. Suitable culture conditions and/or immortalization protocols are known to a person skilled in the art. In addition, a skilled person is aware that different cell lines have different optimal culture conditions. In further embodiments, the living cells may be immortalized cell lines. In certain embodiments, the cell line is an adherent cell line. In alternative embodiments, the cell line is a suspension cell line.

Immortalized cell lines suitable for in vivo testing are well known to a person skilled in the art. The cells may be derived from recognized cell distributors including the American Type Culture Collection (ATCC), the European Collection of Authenticated Cell Cultures (ECACC), Cellosaurus, or commercial vendors. In alternative embodiments, the living cells may be primary cell lines. In certain embodiments, the in vivo testing includes artificial expression of RIPK1 in the whole organism or cell line. It is understood that by “artificial expression”, any RIPK1 expression or RIPK1 expression level deviating from the RIPK1 expression level as observed in cell under physiological conditions or near-physiological conditions, such as cell culture conditions suitable to cultivate and/or proliferate isolated cell lines are intended. Since any deviating expression level is intended, this includes increased or upregulated RIPK1 expression, decreased or downregulated RIPK1 expression, or even a complete absence of RIPK1 expression. In further embodiments, the artificial expression level of RIPK1 in the whole organism or cell line may be inducible or conditional. Means and methods to express proteins, for inhibiting protein expression, the construction of suitable expression vectors, and methods contributing to the establishment of any biological framework comprising cells that mediate expression of one or more genes encoded in such expression vectors are known to a skilled person and have been described in the art on numerous occasions (e.g.

Srinivasan et al., Fundamentals of Molecular Biology, Current Developments in Biotechnology and Bioengineering, 2017). In certain embodiments, the artificially expressed RIPK1 contains additional amino acid residues not comprised in the natural sequence of RIPK1. In further embodiments, the additional amino acid residues not comprised in the natural sequence of RIPK1 may constitute a tag sequence. Said in vivo testing may also include the detection of the phosphorylation state of RIPK1, RIPK3 and/or MLKL, which is a marker for activation state of the respective molecules.

Phosphorylation of either one of said molecules represents an activation of the latter, while a de-phosphorylation of either one of said molecules represents an inhibition of the latter.

“Cell culture” as used herein refers to an in vitro process wherein cells of plant, animal, or human origin are grown and divided under controlled conditions outside their natural environment. Suitable cell culture conditions have been described in the art and are well known to a skilled person. It is understood that culture conditions may vary for different cell types with respect for the following non-limiting parameters: amino acids, carbohydrates, vitamins, minerals, growth factors, hormones, CO₂, O₂, pH, osmotic pressure, and temperature.

The term “primary cell lines” as used herein is indicative for cell lines that have been sampled from tissue and processed to allow culturing in optimized culture conditions. A skilled person is aware that primary cells have in contrast to immortalized cell lines a limited lifespan, and thus can only undergo a limited amount of cell divisions, i.e. be cultured only a limited period of time in vitro. Primary cell lines can be generated by protocols known to a person skilled in the art, or may alternatively be acquired through commercial providers.

In certain embodiments, the method allows to identify RIPK1 modulators that selectively modulate RIPK1 by including a step wherein the candidate compound is screened for its activity to modulate related proteins including but not limited to RIPK3 and/or MLKL. In further embodiments, the step allowing to identify selective RIPK1 modulators may be one or more in silico analyses on three-dimensional structures of the related protein. In alternative embodiments, the step allowing the identification of selective RIPK1 modulators may be one or more experimental procedures.

A non-limiting example of an experimental procedure is comparison of the level of RIPK1 phosphorylation ratio in presence of the candidate RIPK1 modulator with control RIPK1 phosphorylation as reference to the ratio of RIPK2 or RIPK3 phosphorylation in presence of the said candidate RIPK1 modulator with control RIPK2 or RIPK3 phosphorylation as reference. Selective RIPK1 modulators will show a greater difference in RIPK1 phosphorylation levels compared to the RIPK2 or RIPK3 phosphorylation levels. Selective RIPK1 inhibitors will have a lower value for the RIPK1 phosphorylation ratio compared to the RIPK2 or RIPK3 phosphorylation ratio. In yet alternative embodiments, a combination of one or more in silico analyses with one or more experimental procedures is used to determine RIPK1 selectivity of the candidate RIPK1 modulator.

Assessment of the RIPK1 activity may also be performed by monitoring the level of cellular necroptosis upon activation of the RIPK1 pathway in presence of a candidate RIPK1 modulator compared to a reference level of cellular necroptosis in absence of the any RIPK1 inhibitor.

Optionally, the necroptosis level in presence of one or more necrostatins may be used as positive control.

Levels of necroptosis can be monitored using a variety of molecular techniques to detect certain necroptosis hallmarks or “markers” and are known to a person skilled in the art. These assays may rely on flow cytometry, microscopy, or Western blotting, amongst others. A commonly used marker used in the detection of necroptosis is by measurement of the RIPK1, RIPK3 and/or MLKL phosphorylation status (Johnston and Wang, Necroptosis: MLKL polymerization, 2018).

A cell is said to be positive for (or to express or comprise expression of) a particular marker, when a skilled person will conclude the presence or evidence of a distinct signal, e.g., antibody-detectable or detection by reverse transcription polymerase chain reaction, for that marker when carrying out the appropriate measurement, compared to suitable controls. Where the method allows for quantitative assessment of the marker, positive cells may on average generate a signal that is significantly different from the control, e.g., but without limitation, at least about 1.5-fold higher than such signal generated by control cells, e.g., at least about 2-fold, at least about 4-fold, at least about 10-fold, at least about 20-fold, at least about 30-fold, at least about 40-fold, at least about 50-fold higher or even higher.

The expression of the above cell-specific markers can be detected using any suitable immunological technique known in the art, such as immuno-cytochemistry or affinity adsorption, Western blot analysis, FACS, ELISA, etc., or by any suitable biochemical assay of enzyme activity, or by any suitable technique of measuring the quantity of the marker mRNA, such as Northern blotting, semi-quantitative or quantitative RT-PCR. Sequence data for markers in this disclosure are known in the art and can be obtained from public databases such as GenBank (http://www.ncbi.nlm.nih.gov/).

In further embodiments, the method comprises selecting the identified candidate RIPK1 modulators that inhibit RIPK1 activity.

“Inhibition” as used herein refers to the action of inhibiting a process, in the context of this invention inhibition of a molecular process is envisaged. In certain embodiments, the method comprises selecting RIPK1 modulators that partially inhibit RIPK1 activity, i.e. cause an attenuation of the RIPK1 kinase activity. In alternative embodiments the method comprises selecting identified candidate RIPK1 modulators that completely inhibit RIPK1 kinase activity. In further embodiments, the method comprises selecting inhibitors that irreversible bind to the hydrophobic pocket of RIPK1.

In alternative embodiments, the method comprises selecting inhibitors that reversible bind to the hydrophobic pocket of RIPK1. In certain embodiments, the method comprises selecting candidate RIPK1 modulators that attenuate RIPK1 activity. In certain embodiments, the method further comprises in vivo testing of the ability of the identified RIPK1 candidate modulators for inhibiting the activity of RIPK1. In certain embodiments, the method further comprises a ranking step that allows comparison of the ability of the identified candidate RIPK1 modulators to inhibit RIPK1 activity. In further embodiments, the method comprises selecting the identified candidate RIPK1 modulators that reversibly inhibit RIPK1 activity. In alternative embodiments, the method comprises selecting the identified candidate RIPK1 modulators that irreversibly inhibit RIPK1 activity or its downstream targets RIPK3 and/or MLKL.

The terms “reversible inhibition” and “irreversible inhibition” as used herein are commonly used terms to specify characteristics of an enzyme inhibitor. Binding of an inhibitor to an enzyme is reversible or irreversible. Irreversible inhibitors usually react with the enzyme and change it chemically (e.g. via covalent bond formation). These inhibitors modify key amino acid residues needed for enzymatic activity. In contrast, reversible inhibitors bind non-covalently and different types of inhibition are produced depending on whether these inhibitors bind to the enzyme, the enzyme-substrate complex, or both. Methods to measure the dissociation constants of a reversible inhibitor are well known to a person skilled in the art and include but are not limited to isothermal titration calorimetry.

In certain embodiments, the RIPK1 modulator inhibits RIPK1 activity by inhibiting the kinase activity of RIPK1, or its downstream targets RIPK3 and/or MLKL. In further embodiments, the RIPK1 modulator inhibits RIPK1 activity by inhibiting the kinase activity of RIPK1 and structurally impeding the ability of RIPK1 to engage in protein-protein interactions (e.g. with RIPK3). In further embodiments, the RIPK1 modulator inhibits RIPK1 activity by both inhibiting the kinase activity of RIPK1 and structurally impeding the ability of RIPK1 to form the necrosome. In further embodiments, the RIPK1 modulator inhibits RIPK1 activity by inhibiting the kinase activity of RIPK1 and structurally impeding the ability of RIPK1 to bind to MLKL, RIPK2, or RIPK3. In further embodiments, the RIPK1 modulator inhibits RIPK1 activity by trapping said RIPK1 in an inactive conformation.

In further embodiments, the method further comprises selecting identified candidate RIPK1 modulators from steroid compounds. In even further embodiments, the method further comprises selecting identified candidate RIPK1 modulators from estrogens.

The term “steroid” as used herein refers to a biologically active organic compound containing four rings arranged in a specific molecular configuration. Steroids are components of cell membranes that may impact membrane fluidity and may act as signalling molecules. A steroid core structure is composed of seventeen carbon atoms, bonded together in four rings (A-D). Steroids contain three cyclohexane rings (A-C) and one cyclopentane ring (D).

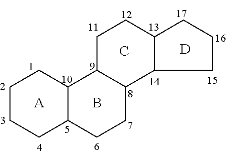

Variation in functional groups attached to the four-ring core and different oxidation states of the rings influence their function. Naturally occurring steroid hormones are synthesized from cholesterol in the gonads and adrenal glands. In certain embodiments, the method selects candidate RIPK1 modulators from animal steroids, human steroids, or a combination hereof. In further embodiments, the method selects candidate RIPK1 modulators from sex hormone steroids, corticosteroids, anabolic steroids, or a combination hereof. In certain embodiments, the method selects candidate RIPK1 modulators from the group of corticosteroids. In certain embodiments, the method selects candidate RIPK1 modulators from the group of sex steroids. In certain embodiments, the method selects candidate RIPK1 modulators from the group of glucocorticoids, mineralocorticoids (corticosteroids), androgens, estrogens, progestogens, or any combination hereof In certain embodiments, the method selects candidate RIPK1 modulators from the group of sterols. In alternative embodiments, the method selects candidate RIPK1 modulators from synthetic steroids. In further embodiments, the method selects candidate RIPK1 modulators from estrogen steroid hormones.

The terms “estrogen”, “estrogenic steroid compound”, “estrogenic compound”, or “oestrogenic steroid compound” as defined herein may be used interchangeably and are indicative for a class of steroid hormones that bind to and activate estrogen receptors. Additionally they may bind to signalling membrane estrogen receptors. Estrogen is the primary female sex hormone and is responsible for regulating the female reproductive system. In addition, it is responsible for the development of secondary sex characteristics. Estrone, estradiol, and estriol are the three most prevalent endogenous estrogens that show estrogenic hormonal activity. Estetrol, a fourth endogenous estrogen is only generated during pregnancy. Estrogens are widely used in contraceptive products and in hormone replacement therapy. It is evident to a skilled person that by estrogens, both naturally occurring estrogens, non-naturally occurring estrogens, metabolic intermediates, and other related compounds such as the non-limiting example of estrogen esters are intended, unless explicitly indicated otherwise.

Also intended are RIPK1 modulators capable of forming a hydrogen bond with amino acid residue Leu 70 of RIPK1 as defined in SEQ ID NO: 1, preferably identified according to any of the methods described herein.

In certain embodiments, the RIPK1 modulator is a naturally occurring molecule. In alternative embodiments, the RIPK1 modulators may be de novo molecules designed in silico. In certain embodiments, the RIPK1 modulators may be a protein domain, a protein fragment, or a peptide. In further embodiments, the RIPK1 modulators may comprise protein domains originating from different naturally occurring proteins. In certain embodiments, the RIPK1 modulators may be steroids. In certain embodiments, the RIPK1 modulator is an animal steroid, preferably a human steroid. In further embodiments, the RIPK1 modulator is a sex hormone steroid. In alternative embodiments, the RIPK1 modulator is a mineralocorticoid (corticosteroid). In alternative embodiments, the RIPK1 modulator is an anabolic steroid. In certain embodiments, the RIPK1 modulator is a sex steroid. In certain embodiments, the RIPK1 modulator is a glucocorticoid. In alternative embodiments, the RIPK1 modulator is an androgen. In alternative embodiments, the RIPK1 modulator is an estrogen. In yet other alternative embodiments, the RIPK1 modulator is a progestogen. In certain embodiments, the RIPK1 modulators may comprise two functionally different domains, wherein one domain binds RIPK1 and a second domain modulates RIPK1 activity.

In certain embodiments, the RIPK1 modulators may contain additional sequences or functional groups not directly involved in binding of RIPK1. In further embodiments wherein the RIPK1 modulator is a protein, a polypeptide or a nucleotide sequence, the RIPK1 modulators may contain sequences encoding additional functionalities to the modulator. Additional RIPK1 modulator functionalities that may be incorporated include but are not limited to RIPK1 localization control, RIPK1 degradation, RIPK1 aggregation, and RIPK1 cellular export. In further embodiments the additional functionalities of the RIPK1 inhibitor are inducible. In even further embodiments the additional functionalities are inducible, whereby non-limiting examples of induction queues include chemical queues, magnetic queues, electrical queues, light queues, or temperature queues. In certain embodiments the modulator may contain additional sequences or functional groups that give temporal and/or spatial control over the RIPK1 inhibitor. In certain embodiments, the RIPK1 modulator may contain additional chemical modifications.

In certain embodiments, the RIPK1 modulator binds at a supplementary location to RIPK1 other than the hydrophobic back pocket, and may therefore be considered an allosteric modulator. In further embodiments, the RIPK1 modulator may be a positive allosteric modulator, negative allosteric modulators, or silent allosteric modulators. In certain embodiments, the RIPK1 modulator binds to both RIPK1 and RIPK2. In alternative embodiments, the RIPK1 modulator binds to both RIPK1, RIPK2, and RIPK3. In yet alternative embodiments, the RIPK1 modulator binds to each of RIPK1, RIPK2, RIPK3.

“Dissociation constant”, or “K_{d}” as used herein is a type of equilibrium constant that indicates the propensity of a larger object to separate, dissociate, reversibly into smaller components. The dissociation constant is the inverse of the association constant. It is known to a person skilled in the art that the dissociation constant is routinely used to quantify the affinity between a ligand and a drug and is therefore indicative for how tightly a ligand binds to a protein. The affinity of a ligand for a protein is influence by the occurrence of, and if present the amount of non-covalent intermolecular interactions between the ligand and the protein such as hydrogen bonds, electrostatic interactions, hydrophobic interactions and Van der Waals forces. In addition, the concentration of other molecules present in the environment the ligand-protein interaction takes place, i.e. macromolecular crowding, can also affect affinities.

In certain embodiments, the RIPK1 modulator forms a hydrogen bond with amino acid residue Leu 70 of RIPK1. In further embodiments, the RIPK1 modulator forms at least one further hydrogen bond(s) with amino acid residue Ile 154 of RIPK1 as defined in SEQ ID NO.1.

In certain embodiments, the RIPK1 modulator further hydrophobically interacts with any one or more of the hydrophobic amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1 in addition to forming a hydrogen bond with Leu 70 or Leu 70 and Ile 154 of said RIPK1.

In certain embodiments the RIPK1 modulator hydrophobically interacts with at least one, at least two, at least three, at least four, at least five, at least six, at least seven, at least eight, at least nine, at least ten, at least eleven, or all twelve of the hydrophobic amino acid residues from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1 in addition to forming a hydrogen bond with Leu 70 of said RIPK1. In certain embodiments the RIPK1 modulator hydrophobically interacts with at least one, at least two, at least three, at least four, at least five, at least six, at least seven, at least eight, at least nine, at least ten, at least eleven, or with all twelve of the hydrophobic amino acid residues from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1 in addition to forming a hydrogen bond with Leu 70 and Ile 154 of said RIPK1.

In further embodiments, the RIPK1 modulator is an RIPK1 inhibitor, or an inhibitor of its downstream targets RIPK3 and/or MLKL.

In certain embodiments the degree of RIPK1 inhibition is dependent on the concentration of RIPK1 inhibitor in the environment the interaction takes place. In certain embodiments, the RIPK1 modulator is a competitive inhibitor.

The term “competitive inhibitor” as used herein is indicative for a reversible RIPK1 inhibitor that mediates interruption of an interaction between two molecules by competing for binding with a ligand of RIPK1. A person skilled in the art is aware of the concept of competitive inhibition, as this has been studied on numerous occasions (e.g. Krohn and Link, Interpreting enzyme and receptor kinetics: keeping it simple, but not too simple. Nuclear medicine and biology, 2003). Chemical inhibition may act on any metabolic or chemical messenger system and may therefore partially or completely interrupt a chemical pathway owing to one chemical substance inhibiting the effect of another. In competitive inhibition in the field of enzyme inhibition, an inhibitor resembling the normal substrate binds to the enzyme, usually at the active site, and will prevent the substrate from binding. In the present invention, it is understood that the hydrophobic pocket of RIPK1 is the active site of the protein, and that the terms “hydrophobic pocket” and “active site” may be used interchangeably herein. In competitive inhibition, the enzyme, here RIPK1, may be bound to the inhibitor, the substrate, or neither. In competitive inhibition, it is not possible that the enzyme, here RIPK1 is bound to both the inhibitor and the natural substrate which both bind the same structural feature. During competitive inhibition, the inhibitor and substrate compete for the active site. The active site is a region on an enzyme which a particular substrate can bind to. Structural limitation of the active site of an enzyme will only allow either binding of the inhibitor or the substrate to bind to the site. As a consequence, the enzyme will be prevented from performing its natural activity when the inhibitor is bound. In competitive inhibition the inhibitor structurally resembles the substrate therefore taking its place. The degree of inhibition of an enzyme in a competitive inhibition system may be subject to the relative concentrations of enzyme, substrate, and inhibitor. Increasing the substrate concentration leads to a loss of competition for the substrate to properly bind to the active site and allow a reaction to occur. In situations where the substrate is available to the enzyme in a higher concentration than that of the competitive inhibitor, it is more likely that the substrate will come into contact with the enzyme, and thus the active site of the enzyme, rather than the inhibitor. Competitive inhibition can be reversible or irreversible. In certain embodiments, the RIPK1 modulator is a reversible competitive RIPK1 inhibitor. In alternative embodiments, the RIPK1 modulator is an irreversible RIPK1 modulator. Competitive inhibition does not impact the maximum velocity of the reaction, but impacts the apparent affinity of a substrate to its binding site on the enzyme.

Competitive inhibition has also been described for inhibitors that bind to an allosteric site of the enzyme. In allosteric competitive inhibition, the inhibitor binds to a site of the enzyme different to that of the active site which prevents substrate binding in the active site. When the substrate is bound to the enzyme, allosteric binding by the inhibitor cannot occur.

In certain embodiments, the RIPK1 inhibitor mediates its effect by sequestering of RIPK1. In alternative embodiments, the RIPK1 inhibitor inhibits RIPK1 by targeting RIPK1 for degradation. In alternative embodiments, the RIPK1 inhibitor inhibits RIPK1 by trapping RIPK1 in an inactive conformation. In an alternative embodiment, the RIPK1 inhibitor inhibits RIPK1 by inducing unfolding of RIPK1. In an alternative embodiment, the RIPK1 inhibitor inhibits RIPK1 by inducing RIPK1 aggregation. In alternative embodiments, the RIPK1 inhibitor inhibits RIPK1 by any combination comprising more than one of the following non-limiting mechanisms: sequestering of RIPK1, targeting RIPK1 for degradation, trapping RIPK1 in an inactive conformation, unfolding RIPK1, inducing RIPK1 aggregation. In further embodiments, the RIPK1 inhibitor mediates its effect in an inducible manner. In alternative embodiments, the RIPK1 modulator mediates its effect in a conditional manner. In certain embodiments, the RIPK1 inhibitor has a potency to inhibit RIPK1, or its downstream targets RIPK3 and/or MLKL, at a level of at least 25%, at least 50%, at least 75%, preferably at least 100%, preferably at least 110%, 115%, 125%, 150%, preferably at least 200% to the level of a known inhibitor such as Necrostatin-1, Necrostatin-1s, necrosulfamide or etanercept.

By the term “conditional manner” as used herein is meant that the RIPK1 modulator or RIPK1 inhibitor only exerts its effects when certain conditions are fulfilled. Non-limiting examples or parameter that may contribute to a condition or a set of conditions that need to be fulfilled include pH, temperature, oxygen level, CO2 level, light, inhibitor concentration, substrate concentration, enzyme concentration, or any combination hereof.

In certain embodiments, the RIPK1 modulator is a steroid compound. In further embodiments, the RIPK1 modulator may be an estrogen.

In certain embodiments, the RIPK1 modulator is a mammalian steroid compound. In further embodiments, the RIPK1 modulator is a sex hormone steroid, a corticosteroid, or an anabolic steroid. In certain embodiments, the RIPK1 modulator is a glucocorticoid, a mineralocorticoid, an androgen, an estrogen, or a progestogen. In certain embodiments, the RIPK1 modulator is a natural steroid compound synthesized from cholesterol. In alternative embodiments, the RIPK1 modulator is a synthetic steroid. In certain embodiments, the RIPK1 modulators may be male or female reproductive hormones. In certain embodiments, the RIPK1 modulator is an estrogen. In further embodiments, the estrogen is selected from the group comprising estradiol, estriol, ethinylestradiol, mestranol and estetrol. In even further embodiments, the RIPK1 modulator is estradiol. In alternative embodiments, the RIPK1 modulator is ethinylestradiol. In other alternative embodiments, the RIPK1 modulator is estriol. In even other alternative embodiments, the RIPK1 modulator is estetrol.

Further intended are pharmaceutical compositions comprising a RIPK1 modulator as described herein for use in modulating the function of RIPK1, or its downstream targets RIPK3 and/or MLKL. The terms “pharmaceutical composition”, “pharmaceutical formulation”, or “pharmaceutical preparation” may be used interchangeably herein and are meant to describe compositions containing a compound of the invention as active pharmaceutical ingredient, formulated with a pharmaceutically acceptable excipient, and manufactured or sold with the approval of a governmental regulatory agency as part of a therapeutic regimen for the treatment of disease in a mammal. It is evident that pharmaceutical compositions are indicative for those compositions that comprise a therapeutically effective amount of the RIPK1 modulator.

The term “therapeutically effective amount” as used herein, refers to an amount of active compound or pharmaceutical agent that elicits the biological or medicinal response in a subject that is being sought by a researcher, veterinarian, medical doctor or other clinician, which may include inter alia alleviation of the symptoms of the disease or condition being treated. Methods are known in the art for determining therapeutically and prophylactically effective doses of pharmaceutical active ingredients or pharmaceutical composition comprising the pharmaceutical active ingredient as taught herein and depend on the RIPK1 modulator, the disease condition and severity, and the age, size and condition of the patient.

“Pharmaceutical active ingredient” or “API” as referred to herein is to be interpreted according to the definition of the term by the World Health organization: a substance used in a finished pharmaceutical product (FPP), intended to furnish pharmacological activity or to otherwise have direct effect in the diagnosis, cure, mitigation, treatment or prevention of disease, or to have direct effect in restoring, correcting or modifying physiological functions in human beings.

“Diagnosis” is indicative for the establishment and conclusion that a subject is affected by a recited disorder. The diagnosis may be based on the examination of symptoms associated with a recited disorder (such as, e.g., clinical diagnosis). Alternatively or in addition, the diagnosis may be made before the symptoms can be examined (i.e., preclinical diagnosis) or because the symptoms are mild or not confined to a recited disorder through, e.g., detecting biomarkers indicative for the recited disorder and/or imaging techniques.

As used herein, a phrase such as “a subject in need of treatment” includes subjects that would benefit from treatment of a given condition, particularly an inflammatory disorder such as neuroinflammatory conditions or diseases. Such subjects may include, without limitation, those that have been diagnosed with said condition, those prone to develop said condition and/or those in who said condition is to be prevented.

The terms “treat” or “treatment” encompass both the therapeutic treatment of an already developed disease or condition, such as the therapy of an already developed (neuro)inflammatory disease, as well as prophylactic or preventive measures, wherein the aim is to prevent or lessen the chances of incidence of an undesired affliction, such as to prevent occurrence, development and progression of a musculoskeletal disease. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. “Treatment” can also mean prolonging survival as compared to expected survival if not receiving treatment.

As used herein, the terms “therapeutic treatment” or “therapy” and the like, refer to treatments wherein the object is to bring a subjects body or an element thereof from an undesired physiological change or disorder, such as a neurological disorder, to a desired state, such as a less severe or unpleasant state (e.g., amelioration or palliation), or back to its normal, healthy state (e.g., restoring the health, the physical integrity and the physical well-being of a subject), to keep it (i.e., not worsening) at said undesired physiological change or disorder (e.g., stabilization), or to prevent or slow down progression to a more severe or worse state compared to said undesired physiological change or disorder.

In certain embodiments, the pharmaceutical formulation further comprises one or more further pharmaceutical active ingredients. In certain embodiments, the pharmaceutical formulation further comprises one or more non-active pharmaceutical ingredients or inactive ingredients, commonly referred to in the art as excipients. In further embodiments, the pharmaceutical composition may be a lyophilized pharmaceutical composition.

The term “excipient”, commonly termed “carrier” in the art may be indicative for all solvents, diluents, buffers (such as, e.g., neutral buffered saline, phosphate buffered saline, or optionally Tris-HCl, acetate or phosphate buffers), solubilisers (such as, e.g., Tween 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, stabilisers, emulsifiers, sweeteners, colorants, flavourings, aromatisers, thickeners, agents for achieving a depot effect, coatings, antifungal agents, preservatives (such as, e.g., Thimerosal™, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of such media and agents for formulating pharmaceutical compositions is well known in the art.

In certain embodiments, the excipient may be an active pharmaceutical ingredient excipient, binder excipient, carrier excipient, co-processed excipient, coating system excipient, controlled release excipient, diluent excipient, disintegrant excipient, dry powder inhalation excipient, effervescent system excipient, emulsifier excipient, lipid excipient, lubricant excipient, modified release excipient, penetration enhancer excipient, permeation enhancer excipient, pH modifier excipient, plasticizer excipient, preservative excipient, preservative excipient, solubilizer excipient, solvent excipient, sustained release excipient, sweetener excipient, taste making excipient, thickener excipient, viscosity modifier excipient, filler excipient, compaction excipient, dry granulation excipient, hot melt extrusion excipient, wet granulation excipient, rapid release agent excipient, increased bioavailability excipient, dispersion excipient, solubility enhancement excipient, stabilizer excipient, capsule filling excipient, or any combination hereof The use of such media and agents for pharmaceutical active substances is well known in the art. Such materials should be non-toxic and should not interfere with the activity of the pharmaceutically active ingredients. In certain embodiments, more than one excipient from the same group is added to the pharmaceutical formulation. In further embodiments, more than one excipient wherein the different excipients belong to different groups is added. In certain embodiments, the excipients may fulfill more than one function.

Furthermore, the formulation may comprise pharmaceutically acceptable auxiliary substances as required to approximate physiological conditions, such as pH adjusting and buffering agents, preservatives, complexing agents, tonicity adjusting agents, wetting agents and the like, non-limiting examples include sodium acetate, sodium lactate, sodium phosphate, sodium hydroxide, hydrogen chloride, benzyl alcohol, parabens, EDTA, sodium oleate, sodium chloride, potassium chloride, calcium chloride, sorbitan monolaurate, and triethanolamine oleate.

In certain embodiments, at least one additional component is combined with the pharmaceutical formulation prior to administration. In further embodiments, the additional component is combined with the pharmaceutical formulation immediately prior to administration. In alternative embodiments where the pharmaceutical formulation is stored under a lyophilized condition, the additional component may be part of the solvent used to reconstitute the formulation. Aqueous solutions suitable for reconstitution of pharmaceutical compositions are known to a person skilled in the art. A non-limiting example of an suitable aqueous solution is water for injection. In certain embodiments, the amount of the additional component added to the pharmaceutical formulation is calculated based on certain patient parameters including but not limited to age, weight, gender, severity of the disease condition, and other known diseases of the patient. In certain embodiments, the additional component may alter bio distribution of the RIPK1 modulator in the body of a subject. In certain embodiments, the additional component is an anti-allergy agent. In alternative embodiments, the additional component is an antiepileptic agent. In alternative embodiments, the additional component may be an analgesic. Non-limiting examples of analgesics or painkillers include paracetamol, nonsteroidal anti-inflammatory drugs (NSAIDS), and opioids. Alternatively, less traditional analgesics may be included in the pharmaceutical compositions described herein such as tricyclic antidepressants and anticonvulsants. In yet alternative embodiments, the additional component may be an anesthetic which has the function to result in a temporary loss of sensation or awareness. In further embodiments, the anesthetic comprised as additional component in the pharmaceutical composition is a local anesthetics. Non limiting examples of local anesthetics include ester local anesthetics such as procaine, amethocaine, cocaine, benzocaine, tetracaine, and amide local anesthetics such as lidocaine, prilocaine, bupivacaine, levobupivacaine, ropivacaine, mepivacaine, dibucaine, etidocaine. Other non-limiting examples of additional components are apoptosis inhibitors; PARP poly(ADP-ribose) polymerase inhibitors; Src inhibitors; agents for the treatment of cardiovascular disorders; anti-inflammatory agents, anti-thrombotic agents; fibrinolytic agents; anti-platelet agents, lipid reducing agents, direct thrombin inhibitors; glycoprotein IIb/IIIa receptor inhibitors; calcium channel blockers; beta-adrenergic receptor blocking agents; cyclooxygenase inhibitors such as COX1 and COX2 inhibitors, angiotensin system inhibitors such as angiotensin-converting enzyme inhibitors; renin inhibitors; and/or agents that bind to cellular adhesion molecules and inhibit the ability of white blood cells to attach to such molecules (e.g., polypeptides, polyclonal and monoclonal antibodies). In certain embodiments the pharmaceutical composition comprises a necrosis inhibitor as additional component. In certain embodiments the pharmaceutical composition comprises a RIPK1 modulator, an apoptosis inhibitor, and a necrosis inhibitor. In certain embodiments the pharmaceutical composition comprises a RIPK1 inhibitor, an apoptosis inhibitor and a necrosis inhibitor.

The term “subject”, “patient”, and “subject in need” may be used interchangeably herein and refer to animals, preferably warm-blooded animals, more preferably vertebrates, and even more preferably mammals specifically including humans and non-human mammals, that have been the object of treatment, observation or experiment. The term “mammals”, or “mammalian subjects” refers to any animal classified as such and include, but are not limited to, humans, domestic animals, commercial animals, farm animals, zoo animals, sport animals, pet and experimental animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Preferred patients are human subjects. Particularly preferred are human subjects, including both genders and all age categories thereof Non-human animal subjects may also include prenatal forms of animals, such as, e.g., embryos or foetuses. Human subjects may also include foetuses, but by preference not embryos.

In certain embodiments, the pharmaceutical composition is a lyophilized composition that may need to be reconstituted prior to administration. In further embodiments, the pharmaceutical composition can be formulated into a unit dosage form, including but not limited to hard capsules, soft capsules, tablets, coated tablets such as lacquered tablets or sugar-coated tablets, granules, aqueous or oily solutions, syrups, emulsions, suspensions, ointments, pastes, lotions, gels, inhalants or suppositories, which may be provided in any suitable packaging means known in the art, non-limiting examples being troches, sachets, pouches, bottles, films, sprays, microcapsules, implants, rods or blister packs.

In certain embodiments, the pharmaceutical composition can be comprised in an implantable dosage form such as a micro container or a microcapsule. In certain embodiments, the pharmaceutical composition is administered systemically. In alternative embodiments, the pharmaceutical composition is administered topically. In certain embodiments, the pharmaceutical combination is used in combinatorial therapy using any known pharmaceutical composition known in the art. In certain embodiments, the pharmaceutical composition is suitable for oral, rectal, bronchial, nasal, topical, buccal, sublingual, transdermal, vaginal or parenteral (including cutaneous, subcutaneous, intramuscular, intraperitoneal, intravenous, intra-arterial, intracerebral, intracerebroventricular intraocular injection or intravenous infusion) administration, or in a form suitable for administration by inhalation or insufflation, including powders and liquid aerosol administration. In embodiments where the pharmaceutical composition is administered parentally, the composition may be comprised in an aqueous solution which is pyrogen-free and has a suitable pH, isotonicity and stability. In those embodiments, the aqueous solution may thus contain sugars, alcohols, antioxidants, buffers, bacteriostats, solutes, suspending agents or thickening agents. In certain embodiments, the pH of the pharmaceutical composition to be administered parentally is adjusted to a physiologic pH in the region of 7 to 9, as found in blood and plasma. In certain embodiments, the pH of the pharmaceutical composition to be administered parentally is from about 7.35 to about 7.45. In certain embodiments, the pharmaceutical composition may be comprised in an immediate release formulation dosage form. In alternative embodiments, the pharmaceutical formulation may be comprised in a delayed release dosage form. In alternative embodiments, the pharmaceutical composition may be comprised in a controlled release formulation dosage form.

The terms “immediate release”, “delayed release”, and “sustained-release” or “controlled release” are clear to a person skilled in the art and are indicative for the release profile of a pharmaceutical composition. In immediate release the pharmaceutical composition is about immediately released from a dosage form to a body of a subject or patient. In delayed release dosage forms, the pharmaceutical composition is delivered in the body with a delay after administration. In sustained release or controlled release dosage forms, the dosage form is designed to release a pharmaceutical composition at a predetermined rate in order to maintain a constant drug concentration for a specific period of time. The release profile of a dosage form can be assessed as described in the major pharmacopeias. For example, immediate release is defined by the European Medicines Agency as dissolution of at least 75% of the active substance within 45 minutes (European Pharmacopeia (Ph. Eur.) 9^{th} edition). However, it is in addition trivial to a person skilled in the art that suitable tests and time windows may vary depending on therapeutic ranges, solubility and permeability factors of the drug substance.

Suitable examples of sustained release systems include semipermeable matrices of solid hydrophobic polymers containing the compound of the invention, which may be in form of shaped articles, e.g. films or microcapsules.

Techniques regarding the formulation and administration of pharmaceutical compositions are known to a skilled person and have been described in the art (e.g. the reference book: Remington: The Science and Practice of Pharmacy, periodically revised).

In further embodiments pharmaceutical compositions comprising a RIPK1 modulator for use in inhibiting the activity of RIPK1 are envisaged.

In certain embodiments, the activity of RIPK1, RIPK3 and/or MLKL is characterized by a linear correlation of the phosphorylation activity of respectively RIPK1, RIPK3 and/or MLKL. In certain embodiments, the pharmaceutical composition comprising a single unit dosage form inhibits the activity of RIPK1, RIPK3 and/or MLKL in a subject, a tissue of the subject, or a cell type of a subject with at least 30%, preferably at least 40%, at least 50%, at least 60%, at least 70%, at least 80%, at least 90%, at least 95%. In certain embodiments, the pharmaceutical composition comprising a single unit dosage form inhibits the activity of RIPK1, RIPK3 and/or MLKL in a tissue of the subject with at least 30%, preferably at least 40%, at least 50%, at least 60%, at least 70%, at least 80%, at least 90%, at least 95%. In certain embodiments, the pharmaceutical composition comprising a single unit dosage form inhibits the activity of RIPK1, RIPK3 and/or MLKL in a cell type of the subject with at least 30%, preferably at least 40%, at least 50%, at least 60%, at least 70%, at least 80%, at least 90%, at least 95%. In certain embodiments, the pharmaceutical composition inhibits the activity of RIPK1, RIPK3 and/or MLKL on a systemic level.

In further embodiments pharmaceutical compositions comprising a RIPK1 modulator for use in inhibiting or preventing necroptosis are envisaged.

In certain embodiments, the pharmaceutical compositions comprise additional biologically active molecules or substances that inhibit or prevent necroptosis. In further embodiments, at least one biologically active molecule or substance that inhibits or prevents necroptosis is contained in the pharmaceutical composition. In certain embodiments, the degree, severity, or percentage of necroptosis may be assessed on the level of the whole organism. In alternative embodiments, the degree, severity, or percentage of necroptosis may be assessed in a specific tissue. In yet alternative embodiments, the degree, severity, or percentage of necroptosis may be assessed in a specific cell type. In further embodiments, the inhibition or prevention of necroptosis is linearly correlated to the amount of pharmaceutical composition administered. In certain embodiments, the degree of inhibition or prevention of necroptosis amounts to at least 25%, preferably at least 50%, at least 60%, at least 70%, at least 75%, at least 80%, at least 85%, at least 90%, at least 95% when compared to an identical situation wherein the pharmaceutical composition was not administered.

In certain embodiments, the pharmaceutical compositions comprising a RIPK1 modulator for use in ameliorating tissue injuries or for blocking necroptotic cell death and inflammation in the treatment of human inflammatory and degenerative diseases are intended. Necroptosis is recognized as an important, drug-targetable contributor to necrotic injury in many pathologies, including ischemia-reperfusion injuries (heart, brain, kidney, liver), brain trauma, eye diseases, and acute inflammatory conditions (Reviewed in Degterev et al. Assays for Necroptosis and activity of RIP kinases, Methods in Enzymology, 2014).

The term “human inflammatory diseases” as used herein refers to diseases characterized by an abnormal inflammation. Non limiting examples of inflammatory disorders include acne vulgaris, allergic reactions, asthma, autoimmune diseases, autoinflammatory diseases, celiac disease, chronic prostatitis, colitis, diverticulitis, glomerulonephritis, hidradenitis suppurativa, hypersensitivities, inflammatory bowel diseases, interstitial cystitis, lichen planus, mast cell activation syndrome, mastocytosis, otitis, pelvic inflammatory disease, reperfusion injury, rheumatic fever, rheumatoid arthritis, rhinitis, sarcoidosis, transplant rejection, vasculitis. Other non-immune diseases originating from inflammatory processes include cancer, atherosclerosis, neurological disorders, ischemic heart disease, (neonatal) hypoxia induced ischemia, (neonatal) hypoxic-ischemic encephalopathy, or any white matter disease/brain injury such as periventricular leukomalacia.

The term “degenerative diseases” as used herein refers to diseases that are characterized by a continuous process based on degenerative cell changes, which eventually affect tissues, cell types, or organs. In certain embodiments, the degenerative diseases are neurodegenerative diseases. In certain embodiments, the neurological disorder is an injury, preferably a central nervous system injury, more preferably a brain injury, or a neurodegenerative disease. In certain embodiments, the neurological disorder is thus selected from the group comprising or consisting of a brain injury, a spinal cord injury, and a neurodegenerative disease. More preferably the neurological disorder is selected from the group comprising or consisting of a brain injury and a neurodegenerative disease.

The group of brain injuries includes, but is by no means limited to, hypoxic/anoxic brain injuries, including hypoxic-ischemic encephalopathy (HIE) such as preferably neonatal HIE, periventricular leukomalacia and further brain ischemia, or stroke, or traumatic brain injuries. In certain embodiments, the brain injury, such as hypoxic brain injury, anoxic brain injury, or traumatic brain injury, affects at least the hippocampus, more preferably the brain injury disrupts brain cell integrity in at least the hippocampus. In certain embodiments, the brain injury affects a brain region selected from but not limited to the group consisting essentially of basal ganglia, basal thalami, subcortical white matter, hippocampus, or any combination thereof.

The terms “hippocampus” or “hippocampal formation” are used as synonyms herein and refer to a brain region located in the medial temporal lobe of the brain that is involved in memory, and spatial memory and navigation. Mammals have two hippocampi, in each side of the brain and the term encompasses both hippocampi. As used herein, hippocampus refers to dentate gyrus (DG), cornu ammonis (CA) and subiculum. The dentate gyrus encompasses the fascia dentata, the hilus, the subgranular zone (SGZ), the granule cell layer, and the molecular layer. The subgranular zone (SGZ) is a narrow layer of cells located between the granule cell layer and the hilus of the DG. Conu ammonis (CA) is differentiated into the fields conu ammonis 1 (CA1), conu ammonis 2 (CA2), conu ammonis 3 (CA3), and conu ammonis 4 (CA4). The neurological disorder may affect at least one, more than one or all of these regions, such as in particular, at least one, more than one or all of dentate gyrus, conu ammonis 1, conu ammonis 2, conu ammonis 3, or subgranular zone.

The terms “cortex” and “cerebral cortex” are used as synonyms herein and generally denote the outermost sheet of neural tissue of the cerebrum. Cortex may be generally seen as composed of sensory, motor, and association areas.

Exemplary injuries to the spinal cord and associated ganglia include, but are not limited to, post-polio syndrome, traumatic injury, surgical injury, or paralytic diseases.

In certain embodiments, the pharmaceutical compositions as disclosed herein may be used to counteract brain damage which may be optionally caused by an inflammatory process, while promoting neurogenesis and vasculogenesis. In certain embodiments, the pharmaceutical compositions as disclosed herein may be used to counteract inflammation, while promoting neurogenesis and vasculogenesis. In further embodiments, the pharmaceutical compositions are administered as early as possible after hypoxia, trauma or hypoxia-ischemia, preferably within about 24 hours, more preferably within about 12 hours, within about 6 hours, within about 5 hours, within about 4 hours, within about 3 hours, within about 2 hours, within about 1.5 hour, within about 1 hour, within about 45 minutes, within about 30 minutes in subjects in need hereof In further embodiments, the pharmaceutical composition is administered during recovery after a surgical intervention. In certain embodiments, the pharmaceutical composition is administered to a subject in need as part of a combinatorial treatment strategy. In certain embodiments, the pharmaceutical composition is administered in combination with induced hypothermia. In further embodiments, the induced hypothermia reduces the body temperature of the subject to between about 33° C. and about 34° C. Further intended is a method of treating or preventing necroptosis comprising administering a RIPK1 modulator or a pharmaceutical composition as disclosed herein, to a subject. Further intended is a method of treating tissue injury, inflammatory diseases, or degenerative diseases comprising administering a RIPK1 modulator or a pharmaceutical composition as disclosed herein to a subject.

In certain embodiments, the method of treatment comprises a continuous administration of the RIPK1 modulator or pharmaceutical composition to the subject, such as but by no means limited to intravenous administration. In certain embodiments, the RIPK1 modulator or pharmaceutical composition as disclosed herein is used in the treatment of a neuroinflammatory disease. In certain embodiments, the RIPK1 modulator or pharmaceutical composition as disclosed herein is used in the treatment of a neurodegenerative disease. In certain embodiments, the RIPK1 modulator or pharmaceutical composition as disclosed herein is used in the treatment of a brain injury including but not limited to brain ischemia, stroke, or traumatic brain injury.

Also intended is the use of a RIPK1 modulator as disclosed herein for the manufacture of a medicament for the prevention or treatment of necroptosis. Furthermore, the use of a RIPK1 modulator as disclosed herein for the manufacture of a medicament for the prevention or treatment of tissue injury, inflammatory diseases, or degenerative diseases is also envisaged.

In certain embodiments, Ethinylestradiol (EE) for use in the treatment of necroptosis is intended. In further embodiments, Ethinylestradiol (EE) for use in the treatment of tissue injury, inflammatory diseases or degenerative diseases is intended. In alternative embodiments, Estradiol (E2) for use in the treatment of necroptosis is intended. In further embodiments, Estradiol (E2) for use in the treatment of tissue injury, inflammatory diseases or degenerative diseases is intended. In alternative embodiments, Estriol (E3) for use in the treatment of necroptosis is intended. In further embodiments, Estriol (E3) for use in the treatment of tissue injury, inflammatory diseases or degenerative diseases is intended. In yet alternative embodiments, Estetrol (E4) for use in the treatment of necroptosis is intended. In yet further embodiments, Estetrol (E4) for use in the treatment of tissue injury, inflammatory diseases or degenerative diseases is intended. In alternative embodiments, combinatorial use of at least one estrogen and at least one necrostatin in the treatment of necroptosis is intended. Also intended is the combinatorial use of at least one estrogen and at least one necrostatin in the treatment of tissue injury, inflammatory diseases or degenerative diseases.

The herein disclosed aspects and embodiments of the invention are further supported by the following non-limiting examples.

### EXAMPLES

### Example 1: Retrieval of RIPK1 3-Dimensional Structure

The 3-dimensional (3D) structure of human RIPK1 was downloaded from the Protein Data Bank (PDB ID: 4ITJ) (Berman et al., The Protein Data Bank, Nucleic Acids Research, 2000; Berger S B, Harris P, Nagilla R, Kasparcova V, Hoffman S, Swift B, Dare L, Schaeffer M, Capriotti C, Ouellette M, King BW (2015) Characterization of GSK' 963: a structurally distinct, potent and selective inhibitor of RIP1 kinase. Cell Death Dis July 27; 1:15009). The structure which was deposited reveals RIPK1 in complex with its known inhibitor necrostatin-4 and contains both chains of the enzyme (Xie et al., Structural basis of RIP1 inhibition by Necrostatins, Structure, 2013). The enzyme was cloned and expressed in a Spodoptera frugiperda expression system. Afterwards, the protein was crystallized and resolved by X-ray diffraction at a resolution of 1.8 Å. USCF chimera was used for energy minimization of the downloaded 3-D structure of RIPK1 (Pettersen et al., Chimera—a visualization system for exploratory research and analysis, Journal of Computational Chemistry). The method involved removal of the ligand 1-HX and the heteroatom iodide from the enzyme structure, followed by energy minimization using steepest descent method for 100 steps (0.02 Å step size) and then by conjugate gradient method which has ten steps with step size of 0.02 Å.

### Example 2: Identification of Binding Residues of Known RIPK1 Modulators by Molecular Docking

Auto Dock Tools 1.5.6 (ADT) (autodock.scripps.edu) was used to perform the docking studies of RIPK1 with candidate modulators. The non-polar hydrogen present in the enzyme were merged and torsions were applied to the ligand by rotation of all the rotatable bonds. The molecule was then assigned Gestgeiger partial charges. Using ADT, polar hydrogen atoms, solvation parameters and Kollman charges were also added to the enzyme. Among the three search algorithm options offered by ADT, Lamarckian genetic algorithm (LGA) was selected to analyze active binding of RIPK1 with various inhibitors. Docking for RIPK1 was performed into a grid box near the enzyme's catalytic site with number of points 88,112 and 100 in the X, Y and Z direction respectively with center grid box value of 25.147, 2.253 and 54.305 for X, Y and Z-center. Spacing for grid box for docking studies of the enzyme was kept at 0.375 Awhile ensuring that all the active site residues are entirely covered by the grid box and the ligands are given space for translational and rotational walk.

For docking study of every ligand, 30 independent runs were performed with maximum number of 27,000 GA operations generated on a single population of 150 individuals. Parameters like rate of crossover, rate of gene mutation, and elitism were set to their default values of 0.80, 0.02, and 1, respectively. For further analysis and visualization of protein-ligand interaction patterns, 2D protein-ligand interaction diagram was generated using the Ligand Interaction script in Maestro (Schrödinger Inc., www.schrodinger.com).

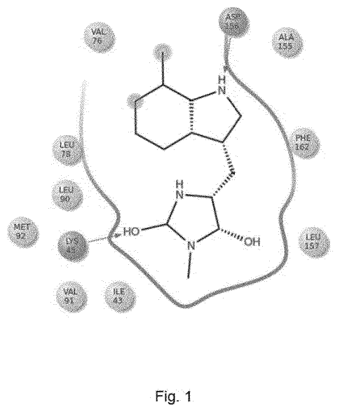

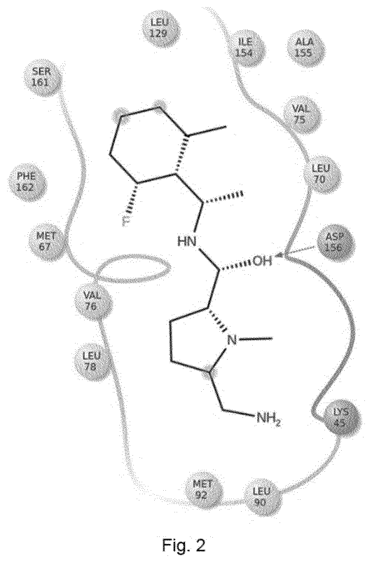

Necrostatin-1 displayed hydrogen bonding with the side chain of amino acid residues Lys 45 and Asp 156 whereas necrostatin-4 exerts a single hydrogen bond with side chain of Asp 156 only. Neither Necrostatin-1 nor Necrostatin-4 displayed hydrogen bond formation with the backbone of any amino acid residue. Binding with Necrostatin-1 is characterized by hydrophobic interaction with Val 76, Leu 78, Leu 90, Val 91, Met 92, Ile 43, Leu 157, Phe 162 and Ala 155 (FIG. 1). Necrostatin-4 RIPK1 interaction is characterized by an elaborate hydrophobic interaction network comprising the polar amino acid Ser 161, the positively charged Lys 45, the hydrophobic residues Phe 162, Met 67, Val 76, Leu 78, Met 92, Leu 90, Leu 70, Val 75, Ala 155, Ile 154 and Leu 129 (FIG. 2).

### Example 3. Identification of Binding Residues of Novel RIPK1 Modulators by Molecular Docking

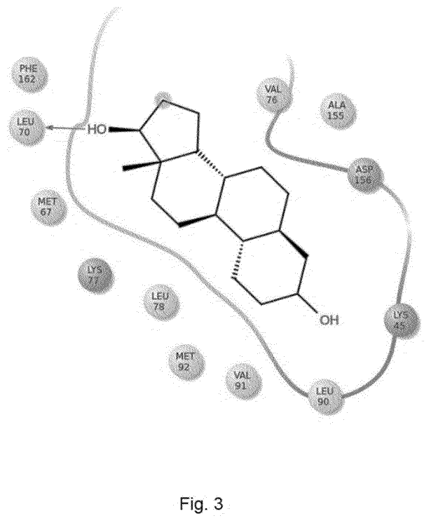

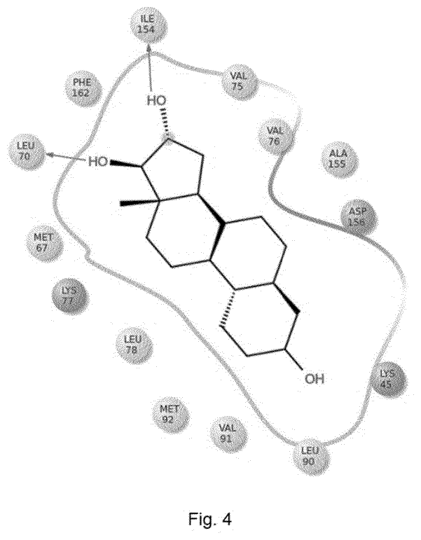

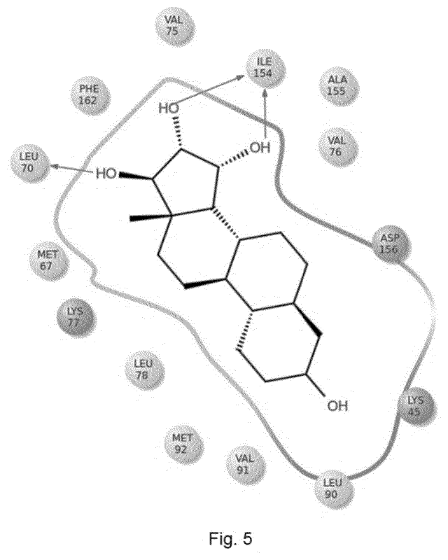

The method described in example 2 was repeated to identify novel interactors of RIPK1. Estradiol shows a single hydrogen bond with Leu 70 amino acid backbone (FIG. 3), whereas Estriol exhibits two hydrogen bonding with backbones of Leu 70 and Ile 154 (FIG. 4). Estradiol hydrophobically interacts with hydrophobic amino acid residues Val 76, Ala 155, Leu 90, Val 91, Met 92, Leu 78, Met 67, and Phe 162. It also interacts with two positively charged Lys residues in position 77 and 45 and one negatively charged Asp 156 residue (FIG. 3). Estriol shows a hydrophobic interaction pattern similar to Estradiol but displays further interaction with another hydrophobic residue Val 75 (FIG. 4). Estetrol manifests the highest number of hydrogen bonds (FIG. 5). It shows three hydrogen bonds, one with Leu 70 backbone and two with backbone of Ile 154. Hydrophobic interaction of Estetrol with the amino acid residues is similar to that of Estriol. The 3 three estrogens are potent candidate RIPK1 modulators as they form hydrogen bonds with critical amino acid residues and manifest elaborate hydrophobic interaction pattern with RIPK1 back pocket residues.

### Example 4. Analysis of Binding Affinities of Identified Candidate RIPK1 Modulators

AutoDock analysis revealed Estradiol as the compound with highest affinity towards RIPK1 since it has lowest binding energy (−9.57 kcal/mol) as compared to the other ligands. Necrostatin-1 and Necrostatin-4 exhibit binding energies of −8.97 kcal/mol and −8.99 kcal/mol, respectively, which are significantly higher compared to the binding energy of Estradiol. Estriol (binding energy −9.38 kcal/mol) and Estetrol (binding energy −9.33 kcal/mol) also show higher affinity towards RIPK1 compared to the Necrostatins. Estrone shows low affinity towards RIPK1 since it has a high binding energy of −7.01 kcal/mol. The lowest binding energy, estimated inhibition constants and amino acids predicted to be participating in hydrogen bonds are listed in Table-2.

TABLE 2
Analysis of AutoDock results for lowest binding
energies, estimated inhibition constant and predicted hydrogen bond
forming residues of RIPK1 with Estrogen hormones.

| | | | | Amino Acids |
|---|---|---|---|---|
| | | | | predicted to be |
| | | | Estimated | participating in |
| | | Lowest Binding | Inhibition | Hydrogen |
| | | Energy | Constant | Bonding with |
| Ligand | PubChem ID | (kcal/mol) | (Ki) | RIPK1 |
| Nec-1 | 2828334 | −8.97 | 266.11 nM | Asn 99, Asp 156, |
| | | | | Leu 78, Lys 45 |
| Nec-4 | | −8.99 | 256.58 nM | Asn 99, Asp 156, |
| | | | | Lys 45, Tyr 94 |
| Estrone | 27125 | −7.01 | 7.34 μM | Asn 148, Glu 96, |
| | | | | Asn 99, Phe 28, |
| | | | | Lys 45 |
| Estradiol | 5757 | −9.57 | 96.11 nM | Leu 70, Met 67 |
| Estriol | 5870 | −9.38 | 133.22 nM | Leu 70, Ile 154, |
| | | | | Met 67 |
| Estetrol | 5756 | −9.33 | 144.17 nM | Leu 70, Ile 154, |
| | | | | Met 67, Val 91, |
| | | | | Phe 28, Tyr 94, |
| | | | | Glu 96, Met 95, |
| | | | | Lys 45 |

### Example 5: In Vitro Validation of in Silico Results Showing Interaction Between Candidate RIPK1 Modulators and RIPK1

Cellular models of necroptosis have been established and are well known to a person skilled in the art (e.g. Degterev et al. Assays for Necroptosis and activity of RIP kinases, Methods in Enzymology, 2014). Commonly used cell lines which can be commercially acquired that are used to study necroptosis include but are not limited to: Kidney cells such as: Mice Tubular cell line (TKPTS), Mice glomerular endothelial cell line (glENDp54-cf. Linkermann et al, Kidney International (2012) 81, 751-761); HK-2 cell line; NRK-52E cell line (cf. Wang et al, Cell Death and Disease (2016) 7, 2125); or primary cultures of mice renal proximal tubular cells (cf. Yanfang Xu et al., 2015, J Am Soc Nephrol 26: 2647-2658); Heart cells such as: primary cultures of cardiomyocytes or H9C2 cell line (cf. Witek et al., Cytotechnology (2016) 68:2407-2415; Shin et al., Molecular Therapy: Nucleic Acids Vol. 14 Mar. 2019, pp 4l38-44Z9; Chenl and Vunjak-Novakovic, Regen Eng Transl Med. 2018 September; 4(3): 142-153); Lung cells such as AECII cells co-cultured with Human vascular endothelial cells (HUVECs) or primary culture of bronchial epithelial cells (cf. Miller and Spence, Physiology (Bethesda). 2017 May; 32(3): 246-260); Liver cells such as primary mouse hepatocyte cells (cf. Lim et al, Journal of General Virology (2014), 95, 2204-2215; Schwabe and Luedde, Nat Rev Gastroenterol Hepatol. 2018 December; 15(12): 738-752); Huh7 cell line; HepG2 cell line (cf. Lin et al, Cell Death Discovery (2016) 2, 16065); Breast cells such as: MCF7 cell line or T47D cell line. Alternatively, Jurkat cells (A3 or FADD-deficient), U-937 cells, (FADD-deficient) MEFs, HT-29 cells, L929 cells, THP-1 cells, RAW 264.7 cells, J77.4 cells, and (primary culture of) neuronal cells can be used. A large number of cell death or necroptosis inducers have been described that are used in such in vitro models, including but not limited to oxidative stress inducers such as H₂O₂, Fas Ligand (FasL), TNFα, 315-01A, TRAIL, TLR3 and TLR4 agonists such as Poly(I:C) and LPS respectively, and interferons (IFNα, IFNβ, and IFNγ). By including agents that counteract the molecular apoptosis machinery (e.g. zVAD.fmk) or on the contrary induce apoptosis (e.g. cycloheximide, cIAP1/2 inhibitors such as SM164, BV6, or compound A, or TAK1 inhibitors such as (5Z)-7-oxozeaenol), the specific cellular model governing necroptosis can be further explored. Reference and control data are generated by including conditions wherein a known necroptosis inhibitor is applied to the cellular system. A non-exhaustive collection of necroptosis inhibitors has been described in the art and include RIPK1 inhibitors such as but not limited to the Necrostatins family such as Nec-1 and Nec-4 and optimized necrostatin variants such as 7-Cl—O-Nec1, Sibriline, as well as Geldanamycin (a Hsp90 inhibitor), RIPK3 inhibitors such as GSK-843 and GSK-872, and inhibitors of MLKL such as necrosulfonamide.

### Example 6. Assays to Monitor and Measure Necroptotic Cell Death

Assays to measure necroptosis have been described in detail in the art (Degterev et al. Assays for Necroptosis and activity of RIP kinases, Methods in Enzymology, 2014). Assays discussed below are performed to compare the identified RIPK1 candidate modulators to known RIPK1 inhibitors such as but not limited to Necrostatin-1 (Nec-1). In accordance to example 5 described above, the cells in this example may in certain conditions be subjected to treatment with apoptosis inhibitors to differentiate between apoptosis and necroptosis pathway involvement in absence or presence of candidate RIPK1 modulators.

6.1. Analysis of Viability with TNFα Using CellTiter-Glo Assay.

Cells are diluted in fresh media at the density of 5×10⁵ cells/mL. 100 μL is plated into each well of a white clear bottom 96-well plate to allow subsequent analysis as well as microscopic observation of the cells. Human TNFα is dissolved in sterile water to a concentration of 100 μg/mL and further diluted to 1 μg/mL in sterile PBS. 1 μL of TNFα is added to the wells to induce necroptosis, and plate is returned into 37° C. incubator for 24 h. 25 μL of reconstituted CellTiter-Glo assay reagent (Promega, G7570) is added into each well and plate is incubated at room temperature on a rocking platform for 10 min. Luminescence (integration time 0.3-1 s) is measured using a platereader, such as Victor3V (Perkin Elmer) or similar. Finally, viability is calculated according to the formula: Viability (%)=(RLU TNFα well/RLU control well)×100%. The viability is a suitable readout for the capacity of the candidate RIPK1 modulator to inhibit necroptosis when compared with known RIPK1 inhibitors such as Nec-1 in presence of apoptosis inhibitors.

6.2. Determination of Specific Cell Death Using Sytox Green Assay.

SYTOX Green is a cell-impermeable dye, which increases fluorescence upon DNA binding. This provides a convenient readout for cell lysis during necroptosis. Cells are seeded into black clear bottom plates in phenol-red-free RPMI1640 media (e.g. Invitrogen, 11835-030), supplemented with 10% FetalPlex serum and 1% antibiotic-antimycotic mix. At a selected time-point (typically 24-48 h), SYTOX Green (e.g. Invitrogen, S7020) is added to the wells at the final concentration of 1 μM. Cells are incubated at 37° C. for 30 min, and fluorescence (green channel, ex. 488 nm, em. 523 nm) is measured using a platereader (1-s integration time). Hence, in presence of apoptosis inhibitors as described above, increased fluorescence serves as a direct readout for necroptosis, and any influence of candidate RIPK1 modulators on necroptosis in comparison to known RIPK1 inhibitors such as Nec-1.

6.3. Annexin V/PI Assay.

The Annexin V/Propidium iodide (PI) assay provides a simple approach to differentiate apoptosis and necroptosis. Annexin V protein binds to phosphatidylserine (PS) exposed in the outer leaflet of plasma membrane of apoptotic cells in a caspase-dependent fashion. This precedes the loss of plasma membrane integrity. Propidium iodide (PI) is a cell-impermeable DNA dye. Thus, the appearance of Annexin V+/PI cells is characteristic for apoptosis. The cells progress to become Annexin V+/PI+due to secondary necrosis. Activation of necroptosis in cells results in the appearance of Annexin V/PI+cells. Overall, this assay provides convenient means to determine the numbers of dead cells and establishes the lack of apoptotic Annexin V+/PI cells in the sample. In a first step, cells are seeded into a 12-well plate (Costar, 3513) at the density of 5×10⁵ cells/mL (2 mL/well, 1×10⁶ cells). Necroptosis is induced by a necroptosis inducer as described in example 5 whereas apoptosis is inhibited by specific inhibitor of apoptosis, for instance zVAD.fmk, as described in example 5, such as H₂O₂+zVAD.fmk. Subsequently, cells are collected by centrifugation for 5 min at 400 g at room temperature and the resulting cell pellet is resuspended in 500 μL of 1× binding buffer (ApoAlert Annexin V kit; Clontech, 630109), followed by centrifugation. Cells are resuspended in 200 μL of 1× binding buffer supplemented with 5 μL of Annexin V-GFP and 10 μL of PI. 4. After 15 min incubation in the dark, cells are further diluted to 500 μL with 1× binding buffer and analyzed by FACS using FL1 (green, Annexin V-FITC) and FL3 (red, PI) channels. Any cell death observed in H₂O₂+zVAD.fmk treated conditions displaying a necrotic morphology confirms necroptosis.

6.4. Analysis of ROS Increase.

Increase in ROS is one of the important features of necroptotic cell death in a number of cell types, such as MEFs and L929 cells (Shindo, Kakehashi, Okumura, Kumagai, & Nakano, 2013; Vanden Berghe et al., 2010). Two sources of increased ROS have been reported: mitochondrial Complex I and NADPH oxidase (Kim, Beg, & Haura, 2013; Vanden Berghe et al., 2010). It should be noted that ROS may not be a universal feature of necroptosis as no increase in ROS accompanies necroptosis in Jurkat cells (Degterev et al., 2005). A number of ROS sensors can be used to measure ROS increase, including CM-H2DCFDA (Invitrogen, cat no. C6827), CellROX sensors (Invitrogen, cat no. C10444), dihydrorhodamine 123 (Invitrogen, cat no. D632), and others. Sensors differ in fluorescence spectra, sensitivity, and repertoire of ROS species detected.

6.5. Mitochondrial Membrane Depolarization.

Change in mitochondrial transmembrane potential is another hallmark of necrosis, in general, and necroptosis, in particular (as discussed in Vanden Berghe et al., Necroptosis, necrosis and secondary necrosis converge on similar cellular disintegration features, Cell death and differentiation, 2010). In this assay, fluorescent probes such as 3,3′-dihexyloxacarbocyanine (DiOC6(3)) is used. The DiOC6(3) containing reagent is added to the cell culture at a final concentration of 40 μM, and cells are incubated for additional 30 min at 37° C. under standard culture conditions. Afterwards, cells are washed once with prewarmed media and are directly analyzed by FACS using FL1 (green) channel or observed using fluorescent microscope. Treatment of cells with apoptosis inhibitors as described above provides a direct readout of necroptosis and enables to compare the efficacy of candidate RIPK1 modulators compared to the activity of known inhibitors such as Nec-1.

6.6. Analysis of TNFα Gene Expression Changes by RT-qPCR.

In addition to activation of cell death RIPK1 activation has been shown to promote TNFα synthesis, which is indicative for the connections between necrotic cell death and inflammation. In multiple cell lines, autocrine TNF signaling is critical for necroptosis activation and therefore TNFα gene expression is an indication for necroptosis. First, cells are seeded into a 12-well plate in 1 mL of media at the density of 1.5-2×10⁵ cells/well. 24 h later, cells are stimulated with 10 ng/mL mouse TNFα, 50 μM zVAD.fmk, and optionally 1 μg/mL cycloheximide for 6-8 h depending on the specific cell line. Next, total RNA is isolated using a commercial kit, for example, Quick-RNA MiniPrep kit (Zymo Research, R1054). RNA concentration is determined based on OD260. Subsequently, cDNA is synthesized using a commercial cDNA kit based on the use of random primers, for example, iScript cDNA synthesis kit (BioRad, 170-8891). 1 μg of total RNA is diluted to 15 μL with RNase-free water and combined with 4 μL of 5× reaction buffer and 1 μL of enzyme mix. Reactions are incubated in a standard PCR machine: 25° C.-5 min, 42° C.-30 min, 85° C.-5 min. Upon completion, reactions are diluted with 30-80 μL of water. qPCRs are set up in duplicate or triplicate for TNFα and 18S (other suitable housekeeping genes include for instance GAPDH and b-actin).

Relative expression of TNFα will be assessed in samples in presence or absence of candidate RIPK1 modulators, optionally by further addition of apoptosis inhibitors to specifically monitor necroptosis.

6.7. Lactate Dehydrogenase Determination

A yet alternative manner to determine necroptosis is the determination of lactate dehydrogenase (LDH) levels, which are elevated if cell death occurs. In order to assess the involvement of necroptosis in damage induced by oxidative stress, the cells are treated with a RIPK1 (candidate) modulator after stimulation with H₂O₂. A reduction of LDH release in presence of the RIPK1 (candidate) modulator proves the involvement of RIPK1 in neuronal death and establishes the RIPK1 candidate inhibitor as a bonafide inhibitor. Suitable colorimetric methods for quantification of LDH activity are commercially available from e.g. Abcam Inc. Cambridge Mass., USA.

### Example 7. In Vitro Model to Assess the Contribution of Necroptosis in Cell Death Following Oxidative Stress

Primary cultures of cells or cell-lines are incubated with H₂O₂ to induce oxidative stress and treated with apoptosis inhibitor z-VAD-fmk and necroptosis inhibitor, Nec-1. The cells are then labeled with AnnexinV-FITC/PI to evaluate proportion of cell death by apoptosis and necroptosis. The experiment can be supplemented by the determination of lactate dehydrogenase (LDH), a measure of cell death that is also used to characterize necroptosis. To understand the involvement of necroptosis in damage induced by oxidative stress, the cells are treated with Nec-1 after stimulation with H₂O₂. The reduction of LDH release by Nec-1 treatment proves the involvement of RIPK1 in neuronal death. Thus taken together, four different conditions can be envisaged as detailed in table 3:

| | | | Candidate RIPK1 |
|---|---|---|---|
| Condition | H₂O₂ | Apoptosis inhibitor | modulator |
| 1 | + | − | − |
| 2 | + | + | − |
| 3 | + | + | + |
| 4 | + | − | + |

Conditions 3 and 4 are repeated for each candidate RIPK1 modulators subject to the invention disclosed herein. This experiment allows to clearly deduct both the involved cell death mechanism(s) and effectiveness of tested RIPK1 modulator, or more specifically RIPK1 inhibitors.

### Example 8. Identification of Molecular Actors Implicated in Oxidative Stress Induced Cell Death

8.1. RT-qPCR Analysis.

After culturing the cells as described in detail in example 7, total RNA is isolated. The expression of the mRNA of RIPK1, RIPK3, MLKL, caspase-8 and caspase-3 is measured by RT-qPCR, a technique which is a standard practice in life science research. High expression of RIPK1, RIPK3, MLKL and caspase-8 and caspase-3 should be observed in the positive control (stimulation only with H₂O₂ and no z-VAD-fmk treatment), whereas cells treated with apoptosis inhibitor, z-VAD-fmk reveal high expression of RIPK1, RIPK3, MLKL and low expression of caspase-8 and caspase-3. The cells treated with z-VAD-fmk and a RIPK1 inhibitor should reveal low expression of all the above-mentioned genes. The experiment is repeated for the RIPK1 candidate modulators subject to the invention disclosed herein.

8.2. Immunoprecipitation of the Necrosome Complex.

After culturing the cells as described in detail in example 7, cells are washed twice with ice-cold PBS and lysed in 0.5-1 mL lysis buffer containing 0.2% (vol/vol) Triton X-100, 150 mM NaCl, 20 mM Tris-HCl (pH 7.4), 1 mM EDTA, 5 mM NaF, 1 mM NaVO₃ (ortho), 1 mM PMSF, and Complete protease inhibitor cocktail (Roche). Cells are incubated on ice for 30 min to 1 h with periodic mixing. Lysates are cleared by centrifugation at 12,000-14,000 rpm in a tabletop 4° C. microcentrifuge for 10-15 min. Protein concentrations are normalized by using a standard protein assays (e.g., Pierce 660 nm Protein Assay kit, 22662). Subsequently, lysates are precleared by incubating with 5-10 μL Protein A/G UltraLink Resin (Thermo Scientific, 53133) at 4° C. for 1 h with gentle rocking. 2 μg of rabbit anti-RIP3 antibody (ProSci, mouse specific, 2283) is incubated with each sample overnight at 4° C. 5-10 μL of Protein A/G UltraLink Resin is added to the lysate and the sample is incubated at 4° C. for 2 h with gentle rocking. Beads are washed four times with lysis buffer, and proteins are eluted by boiling in 1×SDS-PAGE loading buffer. Finally, the whole cell extract samples and immunoprecipitation samples are analyzed by Western blotting using anti-RIPK1 and anti-FADD antibodies. RIPK1 and FADD (unless in FADD-deficient cells) are detected in the treated immunoprecipitation samples in which oxidative stress was induced e.g. with H₂O₂.

Variations of this method may be envisaged by a skilled person by using antibodies to multiple proteins, including FADD, RIPK1, RIPK3, caspase-8, which also indicates necrosome formation. Alternatively, the formation of the necrosome complex can also be assessed by identification of the TNFR1 complex at the plasma membrane by detecting the interaction between RIPK1 or TRADD and TNFR1 by co-immunoprecipitation. TNFR1 complex formation precedes formation of the necrosome.

8.3. Assessment of Necrosome Formation by Fluorescence Microscopy.

An alternative method for detecting necrosome formation is immunofluorescence-based detection using anti-RIPK3 antibodies. While RIPK3 is present as a diffuse cytosolic signal in control cells, activation of necroptosis and formation of the necrosome leads to initial formation of distinct punctae, which continuously enlarges as necroptosis progresses (as described in Sun et al., Mixed lineage kinase domain-like protein mediates necrosis signaling downstream of RIP3 kinase, Cell, 2012). RIPK3 punctae formation is blocked in presence of known RIPK1 inhibitor Nec-1. Any condition where a candidate RIPK1 modulator or inhibitor is added allows assessment of their efficacy relative to known RIPK1 inhibitors. Methods regarding immunofluorescency and imaging have been described in the art and are known to a skilled person (described inter alia in Matos et al., Immunohistochemistry as an Important Tool in Biomarkers Detection and Clinical Practice, Biomarker insights, 2010 and He et al., Receptor interacting protein kinase-3 determines cellular necrotic response to TNF-alpha, Cell, 2009).

### Example 9. Analysis of Recombinant RIPK1 Kinase Activity and Inhibition by RIPK1 Inhibitors

Assays for detecting RIPK1 kinase activity have been described in the art (e.g. Degterev et al. Assays for Necroptosis and activity of RIP kinases, Methods in Enzymology, 2014) and are applicable for assessing the influence of the identified RIPK1 candidates subject of the invention.

9.1. Endogenous RIPK Autophosphorylation Assay

Cells cultured in presence or absence of candidate RIPK1 modulator are lysed in 1 mL of the buffer containing 1% Triton X-100, 150 mM NaCl, 20 mM HEPES, pH 7.3, 5 mM EDTA, 5 mM NaF, 0.2 mM NaVO3 (ortho), and Complete protease inhibitor cocktail (Roche) for 20 min on ice (occasionally mixing side-to-side) and spun down at 14,000 rpm for 10 min at 4° C.

Immunoprecipitation is carried out for 16 h at 4° C. using 1-2 μg of mouse anti-RIP1 antibody (BD Transduction Labs, cat no. 610458) per sample. 10 μL of Protein A+Protein G magnetic Dynabeads (Invitrogen, 10001D and 10003D respectively) are added the following day for 2 h, followed by three washes with lysis buffer and two washes with 20 mM HEPES/0.025% NP-40, pH 7.3. The precipitated RIPK1-antibody complex is cross-linked to the beads by using DMP reagent (Pierce, 21666) according to the protocol provided by the manufacturer. Beads are subsequently resuspended in 9.5 μL of the kinase reaction buffer containing 20 mM HEPES, pH 7.3, 5 mM MnCl2, 5 mM MgCl2, and 0.025% NP-40 and are further incubated with 0.5 μL of inhibitors in DMSO for 10-15 min at room temperature. Reactions are initiated by the addition of 5 μL of 30 μM ATP (Sigma, A7699) and 3 μCi of γ-^{32P}-ATP (Perkin Elmer, BLU002Z250UC), diluted in kinase reaction buffer. Reactions are performed for 30 min at 30° C. with agitation. Finally, reactions are halted by the addition of 5 μL of standard 4×SDS-PAGE loading buffer and heating at 95° C. for 5 min. 15 μL of the supernatant is loaded on an 8% SDS-PAGE gel, and the RIPK1 band is visualized by autoradiography.

9.2. Analysis of Recombinant RIPK1 Kinase Activity

Alternatives to investigate RIPK1 activity rely on the use of recombinant RIPK1 and include the Kinase-Glo assay, the HTRF KinEASE assay, fluorescence polarization assay, and the Thermomelt assay.

### Example 10. Assessment of Candidate RIPK1 Modulator Binding Specificity to RIPK1

The kinase activity of RIPK1 mediates the bifurcation of the cell death pathway into necroptosis wherein the kinase domain is sufficient to induce cell death. Dimerization of RIPK1/RIPK1 kinase domain is necessary for induction of necroptosis. To understand whether the candidate RIPK1 modulators inhibit kinase activity of RIPK1, FADD deficient Jurkat cells are electroporated with pEGFP vector and vectors encoding FKBP12 based dimerization domain fused with full RIPK1 or only kinase domain of RIPK1. The cells are then further treated with dimerizer AP1510, z-VAD.fmk and Nec-1 or candidate RIPK1 modulator and are checked for viability using FACS. The intact live cells are GFP positive and PI negative. Inhibition of RIPK1 by Nec1 or the candidate RIPK1 modulator leads to survival of cells transfected with full RIPK1 or RIPK-1 kinase domain as compared to cells treated only with z-VAD.fmk. This experiment provides insights regarding the specificity of the candidate RIPK1 modulator towards the kinase domain of RIPK1.

### Example 11. Characterization of the Ability of Candidate RIPK1 Modulator to Interfere with Necroptosis in an In Vivo Model

To evaluate if necroptosis is involved in a physiopathologic process of human inflammatory disease or degenerative disease, animal models are used. Animals are divided into 4 groups: (i) a control group; without exposure to cell death inducers, (ii) stimulated group (sham); induction of cell death but receiving no treatment, (ii) an induced necroptosis group; stimulated animals treated with z-VAD-fmk to specifically inhibit apoptosis pathway and (iv) a treated group in which animals are stimulated and treated with z-VAD-fmk combined with Nec-1 or the candidate RIPK1 modulator. At the end of the experimental protocol, the brain is collected for immunohistological analysis of the Cornu Ammonis (CA) areas CA1 and CA2/CA3 regions of the hippocampus. Total proteins are also extracted in the hippocampus region and isolated for western blot analysis. The protein expression profile is analyzed by immunohistology and by western blot (or RIPK1, RIPK3, phospho-RIPK1, phospho-RIPK3, MLKL, phospho-MLKL, caspase-8, caspase-3). Persistence of cell death in the stimulated animals treated z-VAD-fmk treated group proves presence of an alternate caspase-independent cell death pathway. In contrast, inhibition or amelioration of cell death in Nec-1 or candidate RIPK1 inhibitor treated group points at inhibition of the necroptotic pathway. In the stimulated group, higher expression of RIPK1, RIPK3, phospho-RIPK1, Phospho-RIPK3, MLKL, Phospho-MLKL, caspase-8 and caspase-3 are observed.

In the induced necroptosis group, higher expression of RIPK1, RIPK3, phospho-RIPK1, Phospho-RIPK3, MLKL, and/or Phospho-MLKL are observed. In the treated group there is lower expression of the above-mentioned protein profiles.

### Example 12: Effect of Necrostatin 1 (Nec 1) and Estetrol (E4) on Phosphorylation State of MLKL in MCF7 and HT29 Cells Wherein Necroptosis is Induced by TNF-Alpha and z-VAD-fmk

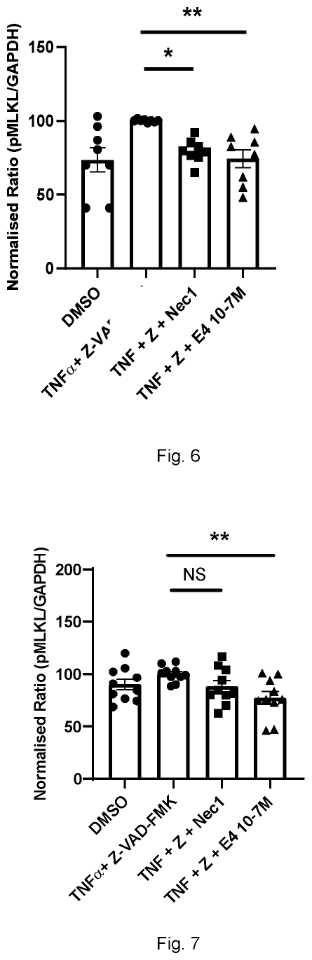

MCF7 and HT29 cells were cultured in DMEM along with 10% FBS, 1% Penicillin-Streptomycin and 1% Glutamine. The cells were pre-treated with 0.1% DMSO or z-VAD-fmk (10 μM) to induce necroptosis (positive control). In order to evaluate the effect of candidate agents on the induced necroptosis, Necrostatin 1 (Nec-1 (10 μM)) or Estetrol (E4 (10-7 M) was added to the cells for 1 hr and was further subjected to Tumor Necrosis Factor (TNF) a (10 ng/ml) treatment for 20 hrs. The cells were then washed, followed by isolation and quantification of the total protein. 20 μg of protein from each treatment group was used for SDS-PAGE separation (10% Agarose gel) and was then transferred to PVDF membrane via a transfer apparatus. Post transfer, the membranes were blocked with 5% milk and further washed with the wash buffer. The membranes were further incubated with anti-Phospho-MLKL (Ser358) Polyclonal Antibody; Cat No: PA5-105678 (Thermofisher Scientific) at a dilution of 1/1000. The membranes were then incubated with secondary antibody (anti-Rabbit IgG, HRP-linked antibody, Cell signaling, Cat No: 7074S) at a dilution of 1/1000 and visualized using Western Blot substrate (Western Lightning Plus-ECL, Perkin Elmer, NEL104001EA) as per manufacturer's protocols. The results are shown in FIGS. 6 and 7, respectively for MCF7 and HT29 cells.

As can be seen from FIGS. 6 and 7, the z-VAD-fmk and TNF-α treated cells (positive control) show an increased phosphorylation of MLKL compared to non-treated cells, indicating that the necroptosis pathway is induced. Cells treated with Necrostatin 1 and estetrol show a decrease of phosphorylation of MLKL versus the positive control. Interestingly, the effect of estetrol surpasses that of Nec1, indicating that the inhibitory effect of estetrol on necroptosis is even better than that of necrostatin 1.

For Nec 1 this reduction in MLKL phosphorylation in MCF7 cells is statistically significant (*p<0.05). for the E4 treated cells, the reduction of MLKL phosphorylation is statistically relevant (**p<0.01). Statistical analysis is done by Ordinary One-Way ANOVA (FIG. 6).

For Nec 1 this reduction in MLKL phosphorylation in HT29 cells is statistically Non-Significant (NS), for the E4 treated cells, the reduction of MLKL phosphorylation is statistically relevant (**p<0.01). Statistical analysis is done by Ordinary One-Way ANOVA (FIG. 7).

1. A method for identifying modulators of Receptor-Interacting Protein Kinase 1 (RIPK1) comprising
a) in-silico analyzing the three-dimensional structure of a candidate molecule and
b) assessing the degree of fit of said three-dimensional structure in the hydrophobic back pocket of RIPK1,
whereby an interaction of said candidate forming a hydrogen bond with amino acid residue Leu 70 of the RIPK1 amino acid sequence as defined in SEQ ID NO.1, indicates the candidate is a modulator of RIPK1, or its downstream targets RIPK3 and/or MLKL.

2. The method according to claim 1, wherein candidate RIPK1 modulators are further selected for their ability to additionally form one or more hydrogen bond(s) with amino acid residue Ile 154 of the RIPK1 amino acid sequence as defined in SEQ ID NO: 1.

3. The method according to claim 1 or 2, wherein candidate RIPK1 modulators are further selected for their ability to hydrophobically interact with any one or more of the hydrophobic amino acid residues selected from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO:1.

4. The method according to any one of claims 1 to 3, further comprising in vivo testing of the ability of the identified candidate modulators for modulating the activity of RIPK1.

5. The method according to any one of claims 1 to 4, wherein said modulators are inhibiting RIPK1 activity, RIPK3 activity and/or MLKL activity.

6. The method according to any one of claims 1 to 5, wherein said candidate modulators are steroid compounds, more preferably estrogens.

7. A RIPK1 modulator capable of forming a hydrogen bond with amino acid residue Leu 70 of the RIPK1 amino acid sequence as defined in SEQ ID NO:1, preferably identified according to the method of any one of claims 1 to 6.

8. The RIPK1 modulator according to claim 7, which additionally forms one or more hydrogen bond(s) with amino acid residue Ile 154 of the RIPK1 amino acid sequence as defined in SEQ ID NO:1.

9. The RIPK1 modulator according to claim 7 or 8, which additionally hydrophobically interacts with any of the hydrophobic amino acid residues from the group consisting of: Val 76, Ala 155, Leu 90, Val 91, Met 92, Leu 78, Met 67, Lys 45, Lys 77, Val 75, Asp 156, or Phe 162 of the RIPK1 amino acid sequence as defined in SEQ ID NO: 1.

10. The RIPK1 modulator according to any one of claims 7 to 9, which is an inhibitor of RIPK1, RIPK3 and/or MLKL.

11. The RIPK1 modulator according to any one of claims 7 to 10, which is a steroid compound, more preferably an estrogen.

12. A pharmaceutical composition comprising the RIPK1 modulator according to any one of claims 7 to 11, for use in modulating the function of RIPK1, RIPK3 and/or MLKL.

13. The pharmaceutical composition for use according to claim 12, for use in inhibiting the activity of RIPK1, RIPK3 and/or MLKL.

14. The pharmaceutical composition for use according to claim 12, for use in inhibiting the phosphorylation of RIPK1, RIPK3 and/or MLKL

15. The pharmaceutical composition for use according to claim 12, for use in inhibiting or preventing necroptosis.

16. The pharmaceutical composition for use according to any one of claims 12 to 15, for ameliorating tissue injuries or for blocking necroptotic cell death and inflammation in the treatment of human inflammatory and degenerative diseases.

17. The pharmaceutical composition for use according to any one of claims 12 to 16, for treating or preventing tissue injury, inflammatory diseases, or degenerative diseases.

18. A method for modulating the function of RIPK1, comprising administering a RIPK1 modulator according to any one of claims 7 to 11, or a pharmaceutical composition according to claim 12 to a subject.

19. A method of treating or preventing necroptosis, comprising administering a RIPK1 modulator according to any one of claims 7 to 11, or a pharmaceutical composition according to claim 12 to a subject.

20. A method of treating or preventing tissue injury, inflammatory diseases, or degenerative diseases, comprising administering a RIPK1 modulator according to any one of claims 7 to 11, or a pharmaceutical composition according to claim 12, to a subject.

21. Use of a RIPK1 modulator according to any one of claims 7 to 11, or a pharmaceutical composition according to claim 12 for the manufacture of a medicament for modulating the function of RIPK1, RIPK3 and/or MLKL.

22. Use of a RIPK1 modulator according to any one of claims 7 to 11, or a pharmaceutical composition according to claim 12, for the manufacture of a medicament for the prevention or treatment of necroptosis.

23. Use of a RIPK1 modulator according to any one of claims 7 to 11, or a pharmaceutical composition according to claim 12, for the manufacture of a medicament for the prevention or treatment of tissue injury, inflammatory diseases, or degenerative diseases.
